(19) United States
(12) Patent Application Publication
(10) Pub. No.: US 2023/0075330 A1
(43) Pub. Date: Mar. 9, 2023

(54) CHIMERIC HIV VIRUS-LIKE PARTICLES

(71) Applicants: ARIZONA BOARD OF REGENTS ON BEHALF OF ARIZONA STATE UNIVERSITY, Scottsdale, AZ (US); FruitVaccine Inc., Champaign, IL (US)

(72) Inventors: Tsafrir S. Leket-Mor, Tempe, AZ (US); Aigerim Kamzina, Tempe, AZ (US); Hugh Mason, Phoenix, AZ (US); Indu Rupassara, Champaign, IL (US)

(21) Appl. No.: 17/822,721

(22) Filed: Aug. 26, 2022

Related U.S. Application Data
(60) Provisional application No. 63/237,847, filed on Aug. 27, 2021.

(51) Int. Cl. C12N 7/00; A61K 39/155; A61P 37/04

(52) U.S. Cl. CPC C12N 7/00; A61K 39/155; A61P 37/04; C12N 2740/16023; C12N 2750/12044; C12N 2740/16034; C12N 2740/16134; C12N 2760/18534; C12N 2740/16171; C12N 2740/16222; A61K 2039/5258

(57) ABSTRACT

Disclosed are compositions and virus-like particles (VLPs) self-assembled from the expression of human immunodeficiency virus (HIV) Gag protein and a fragment of gp41 protein containing its N-terminus ectodomain. The fragment of gp41 protein is linked to an antigen that is not a peptide or protein from HIV, which is presented by HIV VLP. In some aspects, the presented antigen is trimerized. Also disclosed are methods of inducing an immune response against the antigen.

### CROSS-REFERENCE TO RELATED APPLICATIONS

This application claims the benefit of and priority to U.S. provisional patent application 63/237,847, filed Aug. 27, 2021, the entirety of the disclosure of which is hereby incorporated by this reference thereto.

### INCORPORATION-BY-REFERENCE OF MATERIAL ELECTRONICALLY FILED

Incorporated by reference in its entirety herein is a computer-readable nucleotide/amino acid sequence listing submitted concurrently herewith and identified as follows: One 81,994 bytes XML file named “SeqList” created on Aug. 24, 2022.

### TECHNICAL FIELD

The disclosure relates to vaccination compositions and strategies based on virus-like particles formed from human immunodeficiency virus (HIV) Gag protein and a fragment of gp41 protein.

### BACKGROUND

In spite of many advancements in the development of vaccines and various vaccination strategies, successful vaccines against respiratory syncytial virus (RSV) and coronaviruses that cause the common cold, Severe Acute Respiratory Syndrome (SARS), and Coronavirus Disease 2019 (COVID-19) are yet to be developed. Much of the difficulty has been identifying a sufficiently immunologic conserved antigen from these viruses that also trigger the desired immune response for a vaccination. Even for the influenza virus, no long-term vaccine has been developed. Annual seasonal protection still depends on a prediction of the most active subtype in circulation. Accordingly, there is a significant need for vaccine strategies the enhance immunogenicity of viral proteins without causing disease.

### SUMMARY

The disclosure relates to the immunogenicity of an antigen displayed by a virus-like particle (VLP) self-assembled by expression of human immunodeficiency virus (HIV) Gag protein and a fragment of gp41 protein containing the protein's N-terminal ectodomain (for example, a fragment comprising the transmembrane domain and the cytosolic domain). The antigen is not a peptide or protein from HIV and is linked to the fragment of gp41 protein. Thus, the antigen is displayed on the surface of the HIV VLP. In some aspects, the linkage of the antigen and the fragment of gp41 protein resulted in a trimerized antigen being presented by the HIV VLP.

The antigen comprises an ectodomain of a protein and/or a transmembrane domain of a protein. In some aspects, the antigen is a fragment of a transmembrane protein. In particular embodiments, the antigen is a fragment of a protein selected from Influenza HA protein, SARS-CoV Spike (S) protein, SARS-CoV-2 S protein and respiratory syncytial virus (RSV) F protein. In certain embodiments, the antigen is the receptor-binding domain of the S protein of SARS-CoV or SARS-Co-2. In some embodiments, the C-terminus ectodomain of the antigen is linked to the N-terminus of the transmembrane domain of the gp41 protein. In other embodiments, the C-terminus of the transmembrane domain of the antigen is linked to the N-terminus of the cytosolic domain of the gp41 protein.

Also described are plant expression vectors, replicating geminiviral expression systems, and T-DNA binary vectors for producing the described HIV VLPs that present an antigen that is not a peptide or protein from HIV.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts, in accordance with certain embodiments, the structure of the S protein of coronavirus (left) and a schematic of its receptor binding mechanism (right). SARS-CoV-2 recognizes the human angiotensin-converting enzyme 2 on cell surfaces and infects the cells via the receptor binding domain (RBD) of the S protein.

FIGS. 2A-2C depict, in accordance with certain embodiments, a coronavirus particle that causes of severe acute respiratory syndrome (FIG. 2A), a deconstructed HIV VLP produced by coexpression of Gag and a fragment of gp41 (FIG. 2B), and a chimeric SARS-CoV S protein-HIV VLP produced by coexpression of Gag and a fusion protein comprising the S protein ectodomain and the transmembrane and cytosolic domains of gp41 protein. For the VLPs, the lipid membranes are derived from the host cell. In the chimeric SARS-CoV S protein-HIV VLP, the fusion protein of the S protein ectodomain and fragment of gp41 protein forms a trimer, which mimics the presentation of the S protein in the wildtype coronavirus virion.

FIG. 3 depicts, in accordance with certain embodiments, the vector map of pBYEAM-BAF-gp41. Transformation of plant cells with pBYEAM-BAF-produces HIV VLPs that present the respiratory syncytial virus F protein ectodomain at its surface. The HIV VLP is self-assembled from the expression of HIV Gag protein and a fragment of HIV gp41 protein that contains its transmembrane domain and the cytosolic domain.

FIG. 4, depicts, in accordance with certain embodiments, the vector map of pBYKEAM-BAFd27-155C.

FIG. 5 depicts, in accordance with certain embodiments, the vector map of pBYKEAM-BAFd27-DS.

FIG. 6 depict, in accordance with certain embodiments, a schematic representation of particular RSV F constructs. F—native RSV F protein with furin cleavage peptide (p27), transmembrane (TM) and cytosolic domain (CT); Fd27—F with p27 peptide replaced with short linker “GSGSGR” (SEQ ID NO: 1); F-gp41—F protein with its TM/CT replaced with HIV-1 gp41 TM/CT domains which form VLPs when expressed with Gag; Fd27tm-gp41c—F protein with deleted p27 and with its TM/CT fused to gp41 CT; Fd27-gp41—Fd27 with gp41 TM and CT.

FIGS. 7A and 7B depict, in accordance with certain embodiments, the expression of initial RSV F constructs in plants. FIG. 7A show spot infiltration on exemplary Nicotiana benthamiana leaves: 1—F-gp41, 2—Fd27-gp41, 3—F, 4—Fd27. FIG. 7B is an exemplary western blot for total and soluble extracts for expression of corresponding constructs at ˜250 kDa corresponding to trimers.

FIGS. 8A-8C depict, in accordance with certain embodiments, Coomassie staining for F-gp41 co-expressed with Gag to form chimeric VLPs. Samples are taken during extraction and clarification from plants. Lanes from left to right of FIG. 8A are soluble portion of homogenized plant extracts (Sxt), supernatant taken after 20% ammonium sulfate precipitation (AS SN), resuspended pellet from 20% ammonium sulfate precipitation (AS pellet), and AS pellet subjected to ultracentrifugation (fractions include 10%, 20% and 50% of Optiprep). FIGS. 8B and 8C are exemplary western blots probed with F-specific antibody and Gag-specific antibody, respectively.

FIG. 9 depicts, in accordance with certain embodiments, an exemplary dot blot to identify pre-F and total F sites on recombinantly expressed constructs. CR9501 recognizes site Ø that is present on pre-F only, while CR9503 targets site II that is present on both F conformations.

FIG. 10 depicts, in accordance with certain embodiments, a mouse immunization schedule. Six groups of BALB/c mice were immunized with 3 doses of PBS or experimental vaccines with 3 weeks of interval. Subcutaneous (SC) administration included Alum and IN included cholera toxin adjuvant. Serum samples were collected before every injection and 3 weeks after final injection, while nasal flush and fecal samples were collected on the last day after euthanizing.

FIG. 11 depicts, in accordance with certain embodiments, systemic anti-F IgG titers collected throughout the course of the experiment. Each marker represents a single mouse. Arrows show immunizations dates. Data are presented as mean±SD.

FIG. 12 depicts, in accordance with certain embodiments, endpoint titers for anti-F IgG in serum. Terminal bleed mouse sera samples for each group were diluted at a ratio of 1:50 and serially diluted threefold. Each marker represents a single mouse. Data are presented as mean±SD. Statistically significant differences by one-way ANOVA Kruskal-Wallis multiple comparisons test are indicated with an asterisk **, *** indicating p=0.0038, p=0.0002, respectively.

FIG. 13 depicts, in accordance with certain embodiments, the total IgG, IgG2a and IgG1 levels in terminal bleed samples. IgG and its isotypes were determined by ELISA by diluting sera at a ratio of 1:50. Each marker represents a single mouse. Data are presented as mean±SD. Statistically significant differences by one-way ANOVA Kruskal-Wallis multiple comparisons test are indicated with an asterisk **, *** representing p<0.01, p=0.0001, respectively.

FIGS. 14A and 14B depict, in accordance with certain embodiments, the mucosal F-specific IgA titers. FIG. 14A shows IgA titers (by ELISA measurements) from nasal flush obtained from three groups after euthanizing the mice diluted at a ratio of 1:5. FIG. 14B show endpoint titers for pooled fecal samples collected from each group after final vaccine dose was measured with a starting dilution of 1:2. Data are presented as mean±SD.

FIG. 15 depicts, in accordance with certain embodiments, the vector map of pBYKEAM-BAFd27-gp41.

FIG. 16 depicts, in accordance with certain embodiments, the vector map of pBYKEAM-gag.

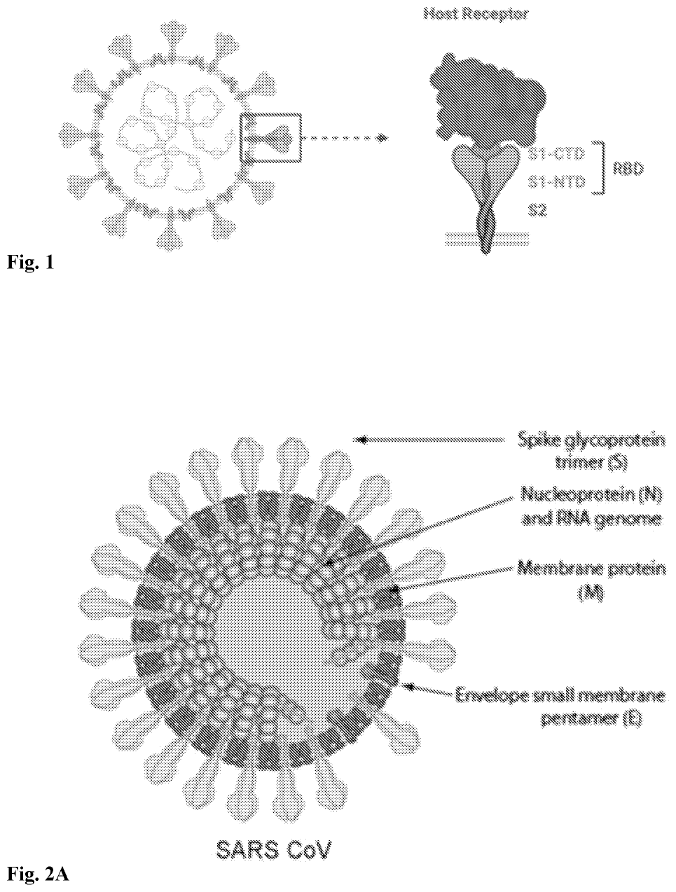

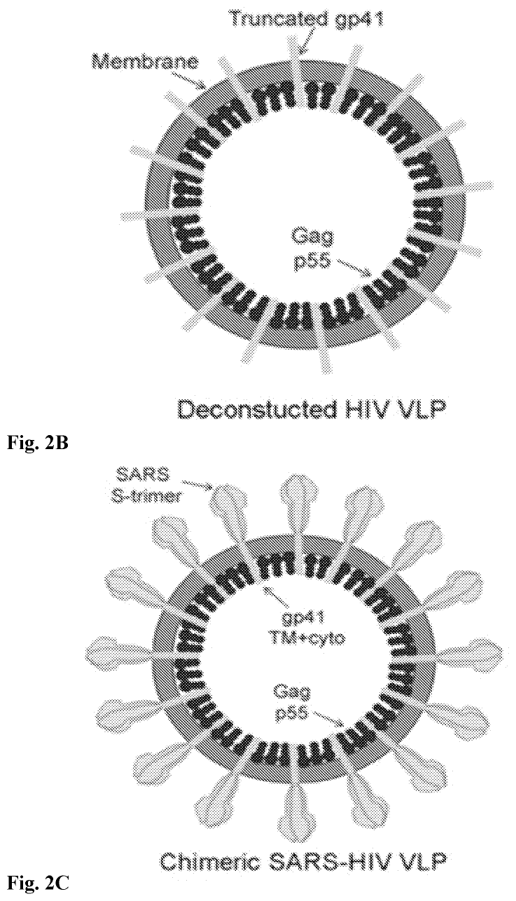

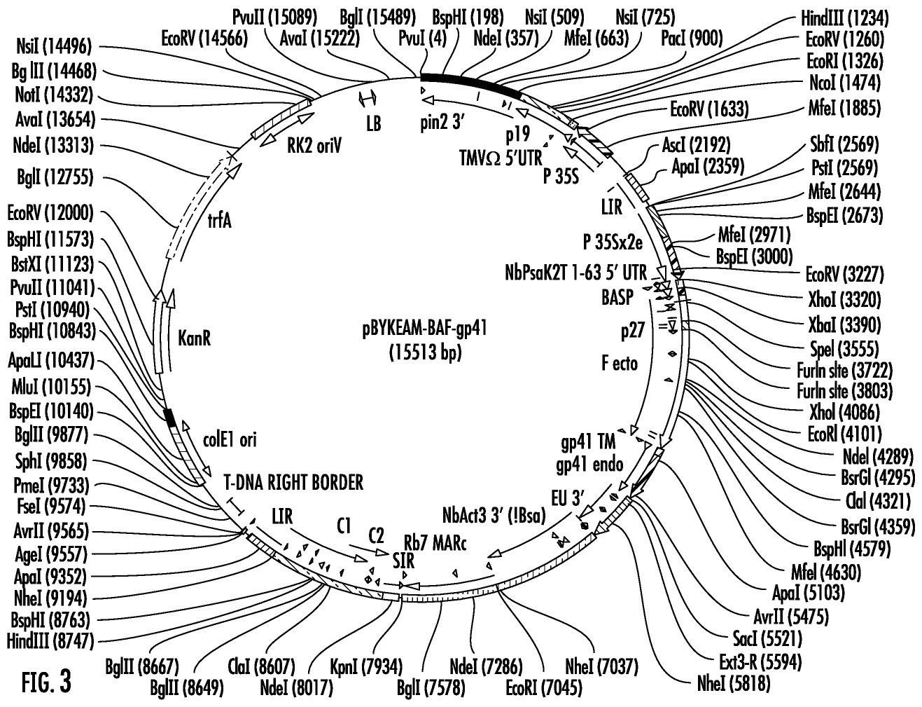

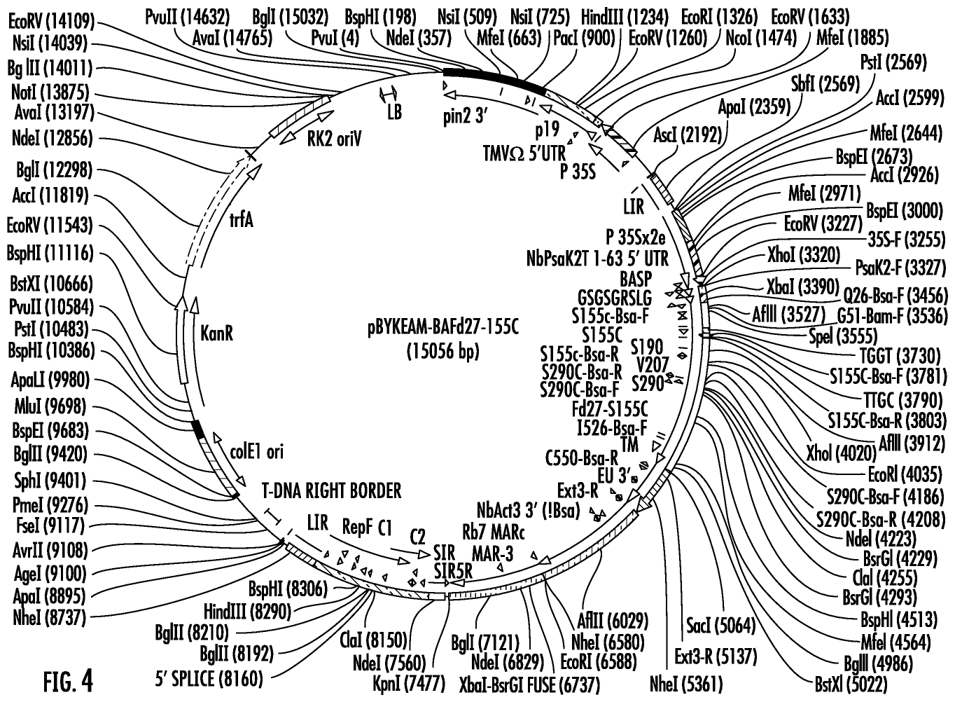

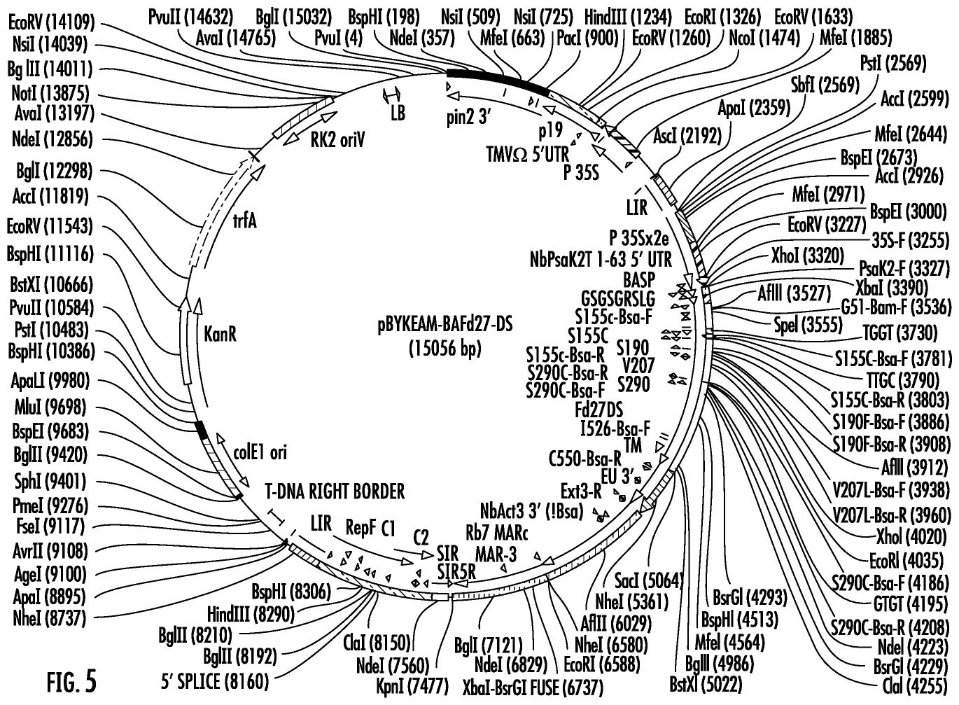

### DETAILED DESCRIPTION

Detailed aspects and applications of the disclosure are described below in the following drawings and detailed description of the technology. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the disclosure. It will be understood, however, by those skilled in the relevant arts, that embodiments of the technology disclosed herein may be practiced without these specific details. It should be noted that there are many different and alternative configurations, devices and technologies to which the disclosed technologies may be applied. The full scope of the technology disclosed herein is not limited to the examples that are described below.

The singular forms “a,” “an,” and “the” include plural referents unless the context clearly dictates otherwise. Thus, for example, reference to “a step” includes reference to one or more of such steps.

As used herein, the term “ectodomain” refers to the domain(s) of a membrane protein that extends into the extracellular space.

As used herein the term “expression vector” refers to a plasmid used to introduce a specific gene, for example a transgene, into a target cell and use the target cell's own mechanism of protein expression to produce the protein encoded by the specific gene. As used herein, the terms “bean yellow dwarf virus vector”, “BeYDV vector,” “BeYDV-based vector,” or a vector of the “BeYDV system” refer to an expression vector that comprises the nucleic acid sequence of BeYDV's long intergenic regions (LIRs) and short intergenic region (SIR). A replication-competent BeYDV vector further comprises the nucleic acid sequence for Rep/RepA.

As used herein, the term “expression cassette” refers to a distinct component of vector DNA, which contains gene sequences and regulatory sequences to be expressed by the transfected cell. An expression cassette comprises three components: a promoter sequence (part of the 5′ untranslated region, 5′ UTR), an open reading frame, and a 3′ untranslated region (3′ UTR). In some aspects, the regulatory sequences are found in the 5′ UTR and the 3′ UTR.

As used herein, the term “fragment” of a protein refers to a peptide or a polypeptide that forms a part of the protein. In some aspects, the fragment of a protein may be one domain of the protein or some of the domains of the protein. For example, a fragment of gp41 may refer to the N-terminal ectodomain of the protein, which in some aspects comprises the membrane-proximal external region (MPER), transmembrane domain, and cytosolic domain of the gp41 protein. As another example, an fragment of the Spike (S) protein of SARS-CoV or SARS-CoV-2 may be the receptor-binding domain of the S protein.

As used herein, the term “replicon” refers to a portion of a vector between two intergenic regions. As used herein, a “mastrevirus replicon” refers to a portion of the vector where two long intergenic region (LTR) from BeYDV designate the borders of the replicon.

As used herein, the term “replicon cassette” refers to an expression cassette comprising at least one gene that assists with replication of an organism. For example, in certain embodiments, the expression vector disclosed herein comprise a replicon cassette comprising a sequence encoding Rep, RepA, or Rep/RepA from BeYDV. A vector that comprises a sequence encoding Rep, RepA, or Rep/RepA from BeYDV is also referred to herein as a vector that is “replication-competent.”

As used herein, the term “replicon vector” refers to a vector that comprises the cis-acting genetic elements necessary to produce replicons. Thus, a replicon vector comprises as its expression cassette a replicon cassette. For example, in certain embodiments, a replicon vector described herein comprises two flanking LIR regions from bean yellow dwarf virus to designate the borders of the replicon. This segment of DNA is amplified via rolling circle replication and other mechanisms by viral and host genes (rep/repA for bean yellow dwarf virus), creating large numbers of DNA copies which serve as transcription templates for the gene of interest in the plant nucleus.

As used herein, the term “terminator” refers to a DNA sequence that contains polyadenylation signals and causes the dissociation of RNA polymerase from DNA and hence terminates transcription of DNA into mRNA. Accordingly, while the term encompasses terminator sequences of known genes, the term also encompasses other sequences that perform the same function, for example, sequences around the short intergenic region of bean yellow dwarf virus.

As used herein, the term “virus-like particle” or “VLP” refers to multiple protein structure that mimic the organization and conformation of authentic native viruses but lack the viral genome. In some embodiments, expression of viral structural proteins, for example capsid or envelope proteins, result in the self-assembly of VLPs.

As referenced herein, the nucleic acid sequence of Gag has at least 95% identity with the nucleic acid sequence of Accession No. AY805330 or Accession No. JX534517. In some embodiments, the nucleic acid sequence of Gag is the optimized nucleic acid sequence encoding Gag for VLP formation previously described in Kessans et al., “Biological and biochemical characterization of HIV-1 Gag/dgp41 virus-like particles expressed in Nicotiana benthamiana.” Plant Biotechnol J. 2013, 11:681-690. The optimized Gag sequence is suitable for VLP formation using a vaccinia virus vector, tobacco mosaic virus-based vector, and geminivirus-based vector. In some aspects, the nucleic acid sequence of the optimized Gag comprises the nucleic acid sequence of Accession No. JX534517.

As referenced herein, the nucleic acid sequence of g41 has at least 95% identity with the nucleic acid sequence of Accession No. AF075722, Accession No. AY805330, or Accession No. JX534518. As used herein, the term “dgp41” refers to a fragment of gp41 protein that is a membrane anchored truncated gp41 presenting the membrane proximal external region with its conserved broadly neutralizing epitopes in the prefusion conformation. In some aspects, the fragment of gp41 comprises the N-terminal ectodomain of the gp41 protein, for example, the transmembrane domain and cytosolic domain of the gp41 protein. In particular embodiments, the fragment of gp41 consists of the transmembrane domain and cytosolic domain of the gp41 protein. For example, the amino acid sequence of the fragment of gp41 is: KIFIMIVGGLIGLR IIFAVLSMVNRVRQGYSPLSFQTLTPNPRGPDRLGRIEEEGGEQDRDRSIRLVSGFLALA WDDLRSLCLFSYHRLRDCILIVARAAELLGRSSLRGLQKGWEALKYLGSLVQYWGLEL KKSAISLLDTTAIAVAEGTDRIIKLIQRICRAICNIPRRIRQGFEAALQ (SEQ ID NO. 2). In other embodiments, the fragment of gp41 comprises the MPER, transmembrane domain, and cytosolic domain of the gp41 protein. In certain embodiments, the fragment of gp41 consists of the MPER, transmembrane domain, and cytosolic domain of the gp41 protein.

The disclosure relates to a vaccination strategy using VLPs formed from Gag protein and a fragment of gp41 protein of human immunodeficiency virus (HIV). VLPs are safe, yet immunogenic, components of several vaccines and candidate vaccines for multiple infectious diseases. The potential for success of VLP-based vaccines is indicated by the widespread use of the human papillomavirus (HPV) vaccines Gardasil© and Cervarix©. To date, many plant-produced vaccines have been tested in animal studies for immunogenicity for both human and veterinary diseases. Additionally, VLPs, but not soluble subunit vaccines, are more likely to properly induce innate and low affinity B cells to transport and display antigen to induce antibody responses.

HIV VLPs have been shown to boost T cell responses following a heterologous prime. CD8 T cell responses, often targeting the Gag protein, are known to be associated with reduced viral load, making this a key target for protective T cell immunity.

The HIV VLP described herein presents an antigen (that is not a peptide or protein from HIV) for the induction of an immune response by linking the antigen to the fragment of gp41. Linking the antigen and the fragment of gp41 forms a fusion protein (also referred to herein as “the gp41 fusion protein”) where the antigen is displayed on the surface of the HIV VLP. As shown in the examples, the HIV VLP described herein induces relatively high systemic and mucosal immune responses.

The antigen is a fragment of protein, for example, a transmembrane protein of a pathogen. In certain implementations, the pathogen is an enveloped virus. In some aspects, the antigen is a fragment of a transmembrane protein that is targeted by neutralizing antibodies. For example, the antigen may be a fragment of Influenza HA protein, SARS-CoV S protein, SARS-CoV-2 S protein, or RSV F protein. In some aspects, the antigen is the ectodomain of a protein such as Influenza HA protein, SARS-CoV S protein, SARS-CoV-2 S protein, or RSV F protein. In other aspects, the antigen comprises the transmembrane domain of a protein such as Influenza HA protein, SARS-CoV S protein, SARS-CoV-2 S protein, or RSV F protein. In some embodiments, the antigen comprises the transmembrane domain and ectodomain of a protein. In particular embodiments, the antigen comprises the ectodomain of the transmembrane protein, for example, the receptor-binding domain of the S protein of SARS-CoV or SARS-CoV-2. In certain implementations, the antigen is one that need to be membrane-embedded and presented in the trimerized form.

In particular embodiments where the antigen is a fragment of RSV F protein, the fragment of RSV F protein lacks the p27 region. In some aspects, the RSV F protein further lacks the cytosolic domain and the transmembrane domain of the RSV F protein. In some implementations, the p27 region is replaced with a linker region comprising the sequence set forth in SEQ ID NO. 2 or a linker region with the sequence of SEQ ID NO. 2. In some aspects, the fragment of RSV F protein comprises at least one point mutation selected from the group consisting of: S155C, S290C, S190F, and V207L.

The fragment of gp41 comprises the N-terminal ectodomain of the protein. In some embodiments, the fragment of gp41 comprises the transmembrane domain and cytosolic domain of the gp41 protein. In some aspects, the fragment of gp41 consists of the transmembrane domain and cytosolic domain of the gp41 protein. In other aspects, the fragment of gp41 is dgp41. In some embodiments, the fragment of gp41 comprises the MPER, transmembrane domain, and cytosolic domain of the gp41 protein. In particular embodiments, the fragment of gp41 consists of the MPER, transmembrane domain, and cytosolic domain of the gp41 protein. Accordingly, in some implementations, the C-terminus ectodomain of the antigen is linked to the N-terminus of the transmembrane domain of the gp41 protein. In other embodiments, the C-terminus of the transmembrane domain of the antigen is linked to the N-terminus of the cytosolic domain of the gp41 protein. In some aspects, linking the fragment of gp41 and the antigen enables the HIV VLP to display the antigen in a trimerized form.

The disclosure is also directed to expression vectors, replicating geminiviral expression systems, and T-DNA binary vectors that produce the above-described HIV VLP. These vectors and expression system enable plant production of HIV VLPs, which offer a number of advantages. The advantages of plant production of recombinant proteins and pharmaceuticals include lack of contamination by mammalian pathogens, less expensive scale-up, speed of expression, and platform flexibility. Many different types of VLPs have been produced in plants. Several plant-made products have been tested in human clinical trials and one product has been approved by the FDA for treatment of Gaucher disease. Economic viability of plant production relies heavily on oftentimes poor yields, and downstream processing costs to remove host proteins accounts for ˜80% of production expenditures.

The HIV VLP described herein can be produced in plants, which provides additional benefits such as, safety from human pathogens, easy scalability, low manufacturing costs, and fast production. Several transient deconstructed viral-based vectors for rapid, high level protein expression in plants are currently available, and all may be used for plant production of the described HIV VLPs. These include the tobacco mosaic virus (TMV)-based magnICON system, which has been extensively used to express recombinant proteins in plants since its invention and was the first to provide gram-levels of antigen, an expression system based on geminiviruses (Gemini), and an expression system based on Cowpea mosaic virus (CPMV)-based pEAQ vectors. The TMV system requires simultaneous delivery of three plasmids by Agrobacterium tumefaciens infiltration (agroinfiltration) to recombine in planta within the nucleus and the TMV movement protein transfers amplified mRNA to surrounding cells. However, Gemini vectors are single plasmid delivery yet lack a movement protein and are known to induce gene silencing, but this can be suppressed using the Tomato bushy stunt virus p19 protein. Furthermore, multiple proteins can be delivered on the same plasmid and expressed in separate replicons established by short and long intergenic regions (SIR/LIR). After transfer to the nucleus, Gemini DNA is amplified via rolling-circle mechanism by the C1/C2 (Rep/RepA) proteins.

HIV VLPs have been produced in plants with low yield, particularly of full-length Gag, being a common theme. Yield can be increased by expressing Gag in transgenic chloroplasts. An efficient production method for enveloped VLPs in the tobacco-relative Nicotiana benthamiana, consisting of Gag and deconstructed-gp4l (dgp41: MPER, transmembrane domain, and full-length cytoplasmic tail) was developed. Such VLPs display the MPER of gp41 without steric hindrance from gp120, without the immunodominant epitopes on both Env subunits, and with a higher antigen load per VLP than what exists in an HIV virion. When administered to mice, these VLPs elicit both serum IgG and mucosal IgA to Gag and dgp41 antigens.

Protein production in plants can be enhanced by presence of 3′ untranslated regions (UTRs) in the expression cassettes of the plant expression vectors. The 5′ UTR and/or the 3′ UTR of the expression cassette may be selected from 5′ UTRs and 3′ UTRs that have been identified to result in enhanced recombinant protein expression in plants (see PCT/US2019/020621, the contents of which are incorporated by reference herein). The 3′ UTR regions that provide enhanced production of the recombinant protein include the extensin 3′ UTR (also referenced herein as the extensin terminator), N. benthamiana actin 3′ UTR (NbACT3), potato proteinase inhibitor II 3′ UTR (Pin2), bean dwarf mosaic virus DNA B nuclear shuttle protein 3′ UTR (BDB), N. benthamiana 18.8 kDa class II heat shock protein 3′ UTR (NbHSP), pea rubisco small subunit 3′ UTR (RbcS), A. thaliana heat shock protein 3′ UTR (AtHSP), cauliflower mosaic virus 35S 3′ UTR (35S), and agrobacterium nopaline synthase 3′ UTR (NOS). The sequences of these 3′UTR are well-known in the art.

In some aspects, the nucleic acid sequence of the extensin terminator is selected from the terminator sequences of the extensin gene in Nicotiana tabacum, Nicotiana tomentosiformis, Nicotiana plumbaginifolia, Nicotinana extensin, Nicotinana sylvestris, Nicotiana benthamiana, Solanum tuberosum, Solanum lycopersicum, Solanum pennellii, Capsicum annuum, and Arabidopsis thaliana, the sequences of which are determinable from GenBank or the Sol Genomics Network. The nucleic acid sequence of the extension terminator comprises a polypurine sequence, an atypical near upstream element (NUE), an alternative polyA site, a far upstream element (FUE)-like region, a major NUE, and a major polyA region, and in certain embodiments, the nucleic acid sequence has at least 72%, at least 73%, at least 74%, at least 75%, at least 76%, at least 77%, at least 78%, at least 79%, 72%, 73%, 74%, 75%, 76%, 77%, 78%, or 79% identity to the sequence of the tobacco (N. tabacum) extension terminator. In some embodiments, the nucleic acid sequence of the extension terminator is that of the tobacco extensin gene. In certain embodiments, the portion of the extensin 3′ UTR in the disclosed vector lacks the intron. In a particular embodiment, the 3′ UTR region of the vector comprises an intronless tobacco extensin terminator (EU). Thus in some aspects, the nucleic acid sequence of EU spans nt) 2764-3126 of the complete N. tabcacum gene for extensin (GenBank D13951.1). In certain other embodiments, the disclosed vector comprises intron-containing extensin terminator. Thus in some aspects, the 3′ UTR region of the vector comprises an intron-containing tobacco extensin terminator (IEU). In such embodiments, the nucleic acid sequence of IEU spans nt 2396-3126 of the complete N. tabcacum gene for extensin (GenBank D13951.1).

In some aspects, the nucleic acid sequence of NbACT3 comprises nt 1460-1853 of actin gene (Gene ID Niben101Scf00096g04015.1). In some aspects, the N. benthamiana actin 3′ UTR is not the entirety of the 3′ UTR, but only the downstream 617-nt region of NbACT3 (NbACT617). In other aspects, the N. benthamiana actin 3′ UTR is not the entirety of the 3′ UTR, but only the downstream 567-nt region of NbACT3 (NbACT567).

In some embodiments, the nucleic acid sequence of Pin2 spans nt 1507-1914 of the potato gene for proteinase inhibitor II (GenBank. X04118.1). In some aspects, the sequence of pinII is obtained from pHB114 (Richter et al., 2000) by SacI-EcoRI digestion.

In some embodiments, the nucleic acid sequence of BDB comprises the 3′ end of the nuclear shuttle protein, the intergenic region, the 3′ end of the movement protein, and additional 200 nt downstream of the movement protein sequence (BDB501), which spans nt 1213-1713 of bean dwarf mosaic virus segment DNA-B (GenBank: M88180.1). In some embodiments, the nucleic acid sequence of BDB comprises only the 282 nucleotides that include the 3′ end of the nuclear shuttle protein, the intergenic region, and the 3′ end of the movement protein (BDB282).

In some embodiments, the nucleic acid sequence of NbHSP comprises the complement to nt 988867-989307 of the sequence of Gene ID Niben101Scf04040. In embodiments, the nucleic acid sequence of NbHSPb comprises the complement to nt 988942-989307 of the sequence of Gene ID Niben101Scf04040.

In some embodiments, the nucleic acid sequence of rbcS comprises a sequence that is complementary to the sequence spanning nt 6-648 of transient gene expression vector pUCPMA-M24 (GenBank: KT388099.1). In some aspects, the sequence of rbcS is obtained from pRTL2-GUS (Carrington et al., 1999) by SacI-EcoRI digestion.

In some embodiments, the nucleic acid sequence of AtHSP comprises nt 1-250 of the partial sequence of the A. thaliana heat shock protein 18.3 gene (GenBank KP008108.1).

In some embodiments, the nucleic acid sequence of 35S comprises a sequence spanning nt 3511-3722 of plant transformation vector pSITEII-8C1 (GenBank: GU734659.1).

In some embodiments, the nucleic acid sequence of NOS comprises nt 22206-22271 of the T-DNA region of cloning vector pSLJ8313 (GenBank: Y18556.1). In some aspects, the sequence of NOS is that of the fragment obtained from pHB103 (Richter et al., 2000) by SacI-EcoRI digestion.

In some embodiments, the 3′ UTR region comprises at least one member from the group consisting of: EU5, IEU, NbACT3, NbACT617, NbACT567, Pin2, BDB501, BDB282, NbHSP, NbHSPb, RbcS, AtHSP, 35S, and NOS. In certain embodiments, the 3′ UTR region of the vector consists of a terminator selected from the group consisting of: EU, NbACT3, Pin2, BDB501, NbHSP, RbcS, NbACT617, NbACT567, NbHSPb, and AtHSP. In some implementations, the 3′ UTR region of the vector consists of a terminator selected from the group consisting of: EU, NbACT3, Pin2, BDB501, NbHSP, and RbcS.

In some aspects, the 3′ UTR comprises two terminators, which produces a double terminator. The double terminator may be a repeat of same terminator or a combination of different terminators (for example, a fusion of two different terminators). In some embodiments, the double terminator consists of EU with NbACT, P19, NbHSP, SIR, NOS, 35S, tobacco mosaic virus 3′ UTR (TMV), BDB501, tobacco necrosis virus-D 3′ UTR (TNVD), pea enation mosaic virus 3′ UTR (PEMV), or barley yellow dwarf virus 3′ UTR (BYDV). In some aspects, the aforementioned pair of terminators are arranged where EU is arranged upstream of the other terminator, which is denoted as EU+NbACT, EU+P19, EU+NbHSP, EU+SIR, EU+NOS, EU+35S, EU+TMV, EU+BDB501, EU+TNVD, EU+PEMV, or EU+BYDV. In some embodiments, the double terminator consists of 35S with NbACT3, NOS, EU, NbHSP, Pin2, or BDB501. In some aspects, the aforementioned pair of terminators are arranged where 35S is arranged upstream of the other terminator, which is denoted as 35S+NbACT3, 35S+NOS, 35S+EU, 35S+NbHSP, 35S+Pin2, or 35S+BDB501. In some embodiments, the double terminator consists of IEU with SIR, 35S, or LIR. In some aspects, the aforementioned pair of terminators are arranged where IEU is arranged upstream of the other terminator, which are denoted as IEU+SIR, IEU+35S, or IEU+LIR. In some embodiments, the double terminator consists of NbHSP with NbACT3, NOS, or Pin2. In some aspects, the aforementioned pair of terminators are arranged where NbHSP is upstream of the other terminator, which is denoted as NbHSP+NbACt3, NbHSP+NOS, or NbHSP+Pin2. In some embodiments, the double terminator consists of NOS with 35S, where NOS is arranged upstream of 35S (NOS+35S).

As used herein, the term “P19” refers to the P19 suppressor of RNAi silencing. An exemplary vector backbone that comprises P19 is pEAQ-HT (see Sainsbury et al., 2009).

In accordance with certain embodiments, the nucleic acid sequence of TMV spans nt 489-693 of the tobacco mosaic virus isolate TMV-JGL coat protein gene (GenBank: KJ624633.1).

In accordance with certain embodiments, the nucleic acid sequence of TNVD has at least 85% identity, preferably 87% identity, to the sequence spanning nt 3457-3673 of the complete genome of tobacco necrosis virus D genome RNA (GenBank: D00942.1). In other embodiments, the nucleic acid sequence of TNVD has at least 90%, preferably 93%, sequence identity with nt 3460-3673 of tobacco necrosis virus-D genome (GenBank: U62546.1).

In accordance with certain embodiments, the nucleic acid sequence of PEMV has at least 95%, preferably 98%, sequence identity with nt 3550-4250 of the pea enation mosaic virus-2 strain UK RNA-dependent RNA-polymerase, hypothetical protein, phloem RNA movement protein, and cell-to-cell RNA movement protein genes (GenBank: AY714213.1).

In accordance with certain embodiments, the nucleic acid sequence of BYDV has at least 95%, preferably 99%, sequence identity with nt 4807-5677 of barley yellow dwarf virus—PAV genomic RNA (GenBank: X07653.1).

In some embodiments, the 5′ UTR comprises the 5′ UTR of native Nicotiana benthamiana NbPsaK, the 5′ UTR from barley yellow mosaic virus, or the 5′ UTR from cowpea mosaic virus. In some aspects, the 3′ UTR comprises the 3′ UTR from barley yellow mosaic virus or the 3′ UTR from cowpea mosaic virus. In certain implementations where the 5′ UTR and the 3′ UTR of the expression cassette is from a virus, the 5′ UTR and the 3′ UTR should come from the same virus, for example if the virus is pea enation mosaic virus. In certain embodiments, the 5′ UTR of the expression cassette does not comprise the 5′ UTR from tobacco mosaic virus or the 5′ UTR from pea enation mosaic virus. In certain embodiments, the 3′ UTR does not comprise the 3′ UTR from pea enation mosaic virus.

The expression level of the expression cassette may also be further enhanced by the selection of a strong promoter, for example, 35S promoter from cauliflower mosaic virus.

In a particular embodiment, the T-DNA region design comprises PinII 3′ UTR, P19, 35S promoter, LIR, NbPsaK truncated 5′ UTR, the transgene, intronless extensin 3′ UTR, NbAct3 3′ UTR, Rb7 MAR, SIR, and Rep/RepA with mutated 5′ UTR. In some aspects, the arrangement of the T-DNA region from 5′ to 3′ is: PinII 3′ UTR—P19—35S promoter—LIR—35S promoter—NbPsaK truncated 5′ UTR—transgene—intronless extensin 3′ UTR—NbAct3 3′ UTR—Rb7 MAR—SIR—Rep/RepA with mutated 5′ UTR—LIR.

In a particular embodiment of the plant expression vector for producing HIV VLP, the T-DNA region in the plant expression vector comprises a 3′ UTR; a suppressor; a promoter; a first long intergenic region; a 5′ UTR; a first nucleic acid sequence encoding Gag protein; a second nucleic acid sequence encoding a fragment of gp41 protein and antigen that is not a peptide or protein from HIV; a short intergenic region; a third nucleic acid sequence encoding Rep/RepA; and a second long intergenic region. The 3′ UTR comprises at least one terminator selected from the group consisting of: EU, IEU, NbACT3, NbACT617 (downstream 617-nt region of NbACT3), NbACT567 (downstream 567nt of NbACT3), Pin2, BDB501, BDB282 (282 nucleotides comprising bean dwarf mosaic virus DNA B nuclear shuttle protein 3′ UTR, the intergenic region, and the 3′ end of the movement protein), NbHSP, NbHSPb (NbHSP missing 75 nt from 5′ end), bean dwarf mosaic virus rep gene 3′ UTR (Rep), pea rubisco small subunit 3′ UTR (RbcS), SIR, SIR 5′/3′ (SIR with additional sequences both upstream and downstream), SIR 3′ (SIR with its additional downstream viral sequence), AtHSP, 35S, bean dwarf mosaic virus repA gene 3′ UTR (RepA), NOS, TMV, TNVD, PEMV, and BYDV.

In some embodiments, the 3′ UTR comprises a combination of (1) a first terminator and a second terminator; (2) a first terminator and a chromatin scaffold/matrix attachment region (MAR); or (3) a first terminator, a second terminator, and a MAR. In some aspects, the first terminator and second terminator form a double terminator. In a particular embodiment, the 3′ UTR comprises the combination of the first terminator, the second terminator, and MAR, wherein the first terminator, the second terminator and MAR are (1) EU, 35S, and Rb7, wherein EU is downstream or upstream of 35S; (2) EU, NbACT3, and Rb7, wherein EU is upstream of NbACT3; (3) EU, BD501, and Rb7, wherein EU is upstream of BD501; (4) EU, A. thaliana heat shock protein 3′ UTR (AtHSP), and Rb7, wherein EU is downstream of AtHSP; (5) EU, 35S, and TM6, wherein EU is upstream of 35S; or (6) IEU, 35S, and Rb7, wherein IEU is upstream of 35S.

In another particular embodiment of the plant expression vector for producing HIV VLP, the T-DNA region in the plant expression vector comprises three expression cassettes. The first expression cassette comprises a sequence encoding Rep and a sequence encoding the promoter of ubiquitin-3 from potato with ubiquitin fusion. The second expression cassette comprises a sequence encoding RepA and a sequence encoding the promoter of ubiquitin-3 from potato with ubiquitin fusion. The third expression cassette comprises a promoter region, a 5′ UTR; a sequence encoding Gag, a sequence encoding a fragment of gp41 and an antigen that is not a peptide or protein from HIV, and a 3′ UTR. In preferred implementations, the expression level of the first expression cassette and the second expression cassette is 1:1.

In yet another particular embodiment of the plant expression vector for producing HIV VLP, the plant expression vector is a T-DNA binary vector. The T-DNA binary vector comprises three expression cassettes. The first expression cassette comprises a sequence encoding Rep anda sequence encoding the promoter of ubiquitin-3 from potato with ubiquitin fusion. The second expression cassette comprises a sequence encoding RepA and a sequence encoding the promoter of ubiquitin-3 from potato with ubiquitin fusion. The third expression cassette comprises a promoter region, a 5′ UTR, a sequence encoding Gag, a sequence encoding a fragment of gp41 and an antigen that is not a peptide or protein from HIV, and a 3′ UTR. In preferred implementations, the expression level of the first expression cassette and the second expression cassette is 1:1.

1. Tobacco Mosaic Virus-Based Vector/Expression System

The tobacco mosaic virus (TMV)-based magnICON system has been extensively used to express recombinant proteins in plants since its invention and was the first to provide gram-levels of antigen. In some aspects, tobacco mosaic virus-based vectors encoding Gag protein and/or the gp41 fusion protein for producing HIV VLP are described. Accordingly, the tobacco mosaic virus-based vector comprises a T-DNA region comprising a nucleic acid sequence encoding Gag protein, gp41 fusion protein, or both the Gag protein and the gp41 fusion protein. In some embodiments, expression of Gag protein, gp41 fusion protein, or a nucleic acid sequence encoding both Gag protein and gp41 fusion protein is driven by the nopaline synthase (NOS) promoter. In some embodiments, expression of Gag protein, gp41 fusion protein, or a nucleic acid sequence encoding both the Gag protein and the gp41 fusion protein is driven by the cauliflower mosaic virus 35S promoter (P35). In some aspects, a nucleic acid sequence encoding an attB site is upstream of the nucleic acid sequence encoding Gag protein, gp41 fusion protein, or both the Gag protein and gp41 fusion protein and is downstream of the promoter.

The TMV system requires simultaneous delivery of three plasmids by Agrobacterium tumefaciens infiltration (agroinfiltration) to recombine in planta within the nucleus and the TMV movement protein transfers amplified mRNA to surrounding cells.

In certain embodiments, the TNDA region comprises two replicons, a first long intergenic region (LIR) from bean yellow dwarf virus (BeYDV), a second LIR for BeYDV; and a third LIR from BeYDV. The first replicon is between the first and the second LIRs from BeYDV, and the second replicon is an expression cassette that is between the second and the third long intergenic regions from BeYDV. The first replicon comprises a first nucleic acid sequence encoding a begomovirus movement protein; a second nucleic acid sequence encoding a begomovirus nuclear shuttle protein; and a first short intergenic region (SIR) from BeYDV. The first SIR separates the first and the second nucleic acid sequences. The second replicon comprises a third nucleic acid sequence encoding Gag protein; a first promoter region upstream of the third nucleic acid sequence; a fourth nucleic acid sequence encoding a fragment of gp41 protein comprising the N-terminal ectodomain of gp41 protein and an antigen that is not a peptide or protein from HIV; and a second promoter upstream of the fourth nucleic acid sequence.

In certain embodiments, the first and second nucleic acid sequences respectively encode the movement protein and the nuclear shuttle protein from the same species of begomovirus. In other embodiments, the first and second nucleic acid sequences do not encode proteins from the same bogomvirus species. For example, in some embodiments, the begomovirus is bean dwarf mosaic virus (BDMV) or abutilon mosaic virus (AbMV). Thus, the first nucleic acid sequence may encodes the movement protein from AbMV, while the second nucleic acid sequence encodes the nuclear shuttle protein from BDMV.

In some aspects, wherein the second replicon further comprises TMV 5′, wherein the TMV 5′ is upstream the third nucleic acid sequence.

In some aspects, the second replicon further comprises a second SIR from BeYDV and a fifth nucleic acid sequence encoding Rep/RepA from BeYDV, wherein the second SIR separates the fourth and fifth nucleic acid sequences and wherein the fifth nucleic acid sequence is downstream of the fourth nucleic acid sequence.

In some aspects, the second replicon further comprises an intronless form of a gene termination from Ext 3′, wherein the Ext 3′ is downstream from the fourth sequence. In certain implementations, the Ext 3′ is upstream of the second SIR.

In particular embodiments, the TATA box of at least one of the first, second, and third long intergenic region is mutated and comprises the nucleic acid sequence TATAAG.

In a particular embodiment, the first replicon further comprises a truncated pinII termination downstream of the second nucleic acid sequence encoding the begomovirus nuclear shuttle protein, wherein the first LIR from BeYDV is a v-sense LIR comprising a mutated TATA box having the sequence TATAAC and is upstream of the second nucleic acid sequence encoding the begomovirus nuclear shuttle protein. The second LIR from BeYDV is a c-sense LIR and is upstream of the first sequence encoding the begomovirus movement protein.

2. Geminivirus-Based Vector/Expression System

In other aspects, a geminivirus-based vector encoding the Gag protein and/or the gp41 fusion protein is used to produce the HIV VLP. Geminivirus-based vectors are single plasmid delivery that lack a movement protein and are known to induce gene silencing, but this can be suppressed using the Tomato bushy stunt virus p19 protein. Furthermore, multiple proteins can be delivered on the same geminivirus-based plasmid and expressed in separate replicons established by short and long intergenic regions (SIR/LIR). After transfer to the nucleus, Gemini DNA is amplified via rolling-circle mechanism by the C1/C2 (Rep/RepA) proteins.

In some embodiments, the geminivirus-based expression system comprises a geminivirus-based vector that encodes the Gag protein and another geminivirus-based vector that encodes the gp41 fusion protein. In other embodiments, the geminivirus-based expression system comprises a geminivirus-based vector that encodes the Gag protein and the gp41 fusion protein and at least one other geminivirus-based vector that modulates the transgene expression, for example a vector that encodes encoding Rep/RepA, begomovirus movement protein, and/or begomovirus nuclear shuttle protein.

In some aspects, the geminivirus-based vector is a vector based on bean yellow dwarf virus (BYDV). Accordingly, the geminivirus-based vector comprises a T-DNA region comprising at least one replicon gene and a nucleic acid sequence encoding the Gag protein and/or a nucleic acid encoding the gp41 fusion protein, wherein the at least one replicon gene is downstream of the nucleic acid sequence encoding the Gag protein and the nucleic acid encoding the gp41 fusion protein. Where the T-DNA region of the geminivirus-based vector comprises a nucleic acid sequence encoding the Gag protein and a nucleic acid encoding the gp41 fusion protein, in some embodiments, the nucleic acid sequence encoding the gp41 fusion protein is upstream of the nucleic acid sequence encoding the Gag protein. In some embodiments, the T-DNA region comprises a nucleic acid encoding Rep/RepA. In certain embodiments, the BYDV short intergenic region is upstream of Rep/RepA.

In some embodiments of the geminivirus-based vector, Gag protein and gp41 fusion protein expression are under the control of P35. Thus, in certain embodiments of the geminivirus-based vector, the T-DNA region further comprises a promoter upstream of the nucleic acid sequence encoding the Gag and the nucleic acid encoding the gp41 fusion protein, where the promoter region comprises a nucleic acid sequence encoding P35. In some aspects, the promoter region further comprises two translation enhancer binding sites (2e), which are downstream of the nucleic acid sequence encoding P35. In certain embodiments, the geminivirus-based vector further comprises a barley α-amylase signal peptide upstream of the nucleic acid encoding the gp41 fusion protein but downstream of the promoter region.

In some aspects, the T-DNA region of the geminivirus-based vector also comprises two BYDV long intergenic regions. One long intergenic region is upstream of the promoter region, and one long intergenic region is downstream of the at least one replicon gene. Where the T-DNA region of the geminivirus-based vector comprises a nucleic acid sequence encoding the Gag protein controlled by one promoter region and a nucleic acid encoding the gp41 fusion protein controlled by another promoter region, the long intergenic regions flank the portion of the T-DNA region containing the nucleic acid sequence encoding the Gag protein, the nucleic acid encoding the gp41 fusion protein, and the at least one replication gene.

In particular embodiments, the T-DNA region of the geminivirus-based vector further comprises a nucleic acid sequence encoding a silencing suppressor protein. In some aspects, the silencing suppressor protein is p19, for example, tomato bushy stunt virus p19. The expression of the silencing suppressor protein by the geminivirus-based vector delays the onset of plant cell necrosis when compared with plant cells transformed with geminivirus-based vectors lacking a nucleic acid sequence encoding the silencing suppressor protein. The nucleic acid sequence encoding the silencing suppressor protein is upstream of the nucleic acid sequence encoding the Gag protein and the nucleic acid encoding the gp41 fusion protein and is upstream of the region flanked by the long intergenic regions. In some aspects, expression of silencing suppressor protein is controlled by a promoter downstream of the nucleic acid sequence encoding the silencing suppressor protein. In certain embodiments, the promoter is NOS.

In particular embodiments, the geminivirus-based expression vector comprises a T-DNA region that comprises two replicons; a first long intergenic region from bean yellow dwarf virus (BeYDV); a second long intergenic region for BeYDV; and a third long intergenic region from BeYDV, wherein the first replicon is between the first and the second long intergenic regions from BeYDV and the second replicon is an expression cassette that is between the second and the third long intergenic regions from BeYDV. The first replicon comprises a first nucleic acid sequence encoding a begomovirus movement protein; a second nucleic acid sequence encoding a begomovirus nuclear shuttle protein; and a first short intergenic region from BeYDV, wherein the first short intergenic region separates the first and the second nucleic acid sequences. The second replicon comprises a third nucleic acid sequence encoding Gag; a first promoter region upstream of the third nucleic acid sequence; a fourth nucleic acid sequence encoding a fragment of gp41 comprising the N-terminal ectodomain of gp41 and an antigen that is not a peptide or protein from HIV; and a second promoter upstream of the fourth nucleic acid sequence.

In some aspects, the begomovirus is bean dwarf mosaic virus (BDMV) or abutilon mosaic virus (AbMV). In some aspects, the TATA box of at least one of the first, second, and third long intergenic region is mutated and comprises the nucleic acid sequence TATAAG.

In some aspects, the first and second nucleic acid sequences respectively encode the movement protein and the nuclear shuttle protein from the same species of begomovirus. In some embodiments, the first nucleic acid sequence encodes the movement protein from AbMV and the second nucleic acid sequence encodes the nuclear shuttle protein from BDMV.

In some embodiments, the second replicon further comprises a second short intergenic region from BeYDV and a fifth nucleic acid sequence encoding Rep/RepA from BeYDV. The second short intergenic region separates the fourth and fifth nucleic acid sequences and wherein the fifth nucleic acid sequence is downstream of the fourth nucleic acid sequence. The second replicon may further comprise 5′ UTR from tobacco mosaic virus (TMV 5′), and the TMV 5′ is upstream the third nucleic acid sequence. The second replicon also may further comprise an intronless form of a gene termination from tobacco extension (Ext 3′), where the Ext 3′ is downstream from the fourth sequence. In some aspects, the Ext 3′ is upstream of the second short intergenic region.

In some embodiments, the first replicon further comprises a truncated pinII termination downstream of the second nucleic acid sequence encoding the begomovirus nuclear shuttle protein. The first long intergenic region (LIR) from BeYDV is a v-sense LIR comprising a mutated TATA box having the sequence TATAAC and is upstream of the second nucleic acid sequence encoding the begomovirus nuclear shuttle protein. The second LIR from BeYDV is a c-sense LIR and is upstream of the first sequence encoding the begomovirus movement protein.

In certain embodiments, the geminivirus-based vector a replicating geminiviral expressing system comprising three cloning vectors, each containing a T-DNA region. The T-DNA region of the first cloning vector comprises a sequence encoding Rep and a sequence encoding the promoter of ubiquitin-3 from potato with ubiquitin fusion. The T-DNA region of the second cloning vector comprises a sequence encoding RepA and a sequence encoding the promoter of ubiquitin-3 from potato with ubiquitin fusion. The T-DNA region of the third cloning vector comprises an expression cassette and no replicon cassette. The expression cassette comprises: a promoter region; a 5′ UTR; a sequence encoding Gag; a sequence encoding a fragment of gp41 and an antigen that is not a peptide or protein of HIV; and a 3′ UTR. In some implementations, the expression level of the first cloning vector and the second cloning vector is 1:1. In other embodiments, the geminiviral-based vector is a T-DNA binary vector comprising three expression cassettes. The first expression cassette comprises a sequence encoding Rep and a sequence encoding the promoter of ubiquitin-3 from potato with ubiquitin fusion. The second expression cassette comprises a sequence encoding RepA and a sequence encoding the promoter of ubiquitin-3 from potato with ubiquitin fusion. The third expression cassette comprises: a promoter region; a 5′ UTR; a sequence encoding Gag; a sequence encoding a fragment of gp41 protein and an antigen that is not a peptide or protein from HIV; and a 3′ UTR.

In some aspects, the sequence encoding Rep and RepA has a mutation in the initiation site at position −3, and the nucleic acid at position −3 is not A or G. For example, the nucleic acid at position −3 is T or C. In certain embodiments, the initiation site sequence of the mutated rep gene or repA gene is CACATG. In other embodiments, the initiation site sequence of the mutated rep gene or repA gene is TACATG. In some embodiments, the rep gene or the repA gene is from bean yellow dwarf virus. In some aspects, the nucleic acid sequence of the repA gene has at least 80% similarity, at least 85% similarity, at least 90% similarity, at least 95% similarity, at least 97% similarity, at least 98% similarity, or at least 99% similarity with the sequence spanning position 1308 to 2398 of GeneBank Y11023.2. In some aspects, the nucleic acid sequence of the rep gene has at least 80% similarity, at least 85% similarity, at least 90% similarity, at least 95% similarity, at least 97% similarity, at least 98% similarity, or at least 99% similarity with the sequence spanning position 1308 to 1519 of GeneBank Y11023.2.

In certain embodiments, the replicating geminiviral expression system for producing a HIV VLP comprises a three cloning vectors, each with a T-DNA region. The T-DNA region of the first cloning vector comprises a sequence encoding Rep anda sequence encoding the promoter of ubiquitin-3 from potato with ubiquitin fusion. The T-DNA region of the second cloning vector comprises a sequence encoding RepA and a sequence encoding the promoter of ubiquitin-3 from potato with ubiquitin fusion. The T-DNA region of the third cloning vector comprises an expression cassette and no replicon cassette. The expression cassette comprises a promoter region; a 5′ UTR; a sequence encoding Gag; a sequence encoding a fragment of gp41 and an antigen that is not a peptide or protein of HIV; and a 3′ UTR. In preferred implementations, the expression level of the first cloning vector and the second cloning vector is 1:1.

Also disclosed are methods of generating an immune response, including a mucosal immune response, against a pathogen where an antigen originating from the pathogen is presented by the disclosed HIV VLP. The method comprises administering the HIV VLP to the subject whether subcutaneously or intranasally. Intranasal administration is preferred against a pathogen that causes respiratory infections. In preferred implementations, the HIV VLP is administered at least twice with each administration separated by at least three weeks. The HIV VLP may be administered as a composition comprising an adjuvant (for example, Alum or cholera toxin).

Illustrative, Non-Limiting Examples in Accordance with Certain Embodiments

The disclosure is further illustrated by the following examples that should not be construed as limiting. The contents of all references, patents, and published patent applications cited throughout this application, as well as the figures, are incorporated herein by reference in their entirety for all purposes.

### I. Vector Construction: RSV VLP

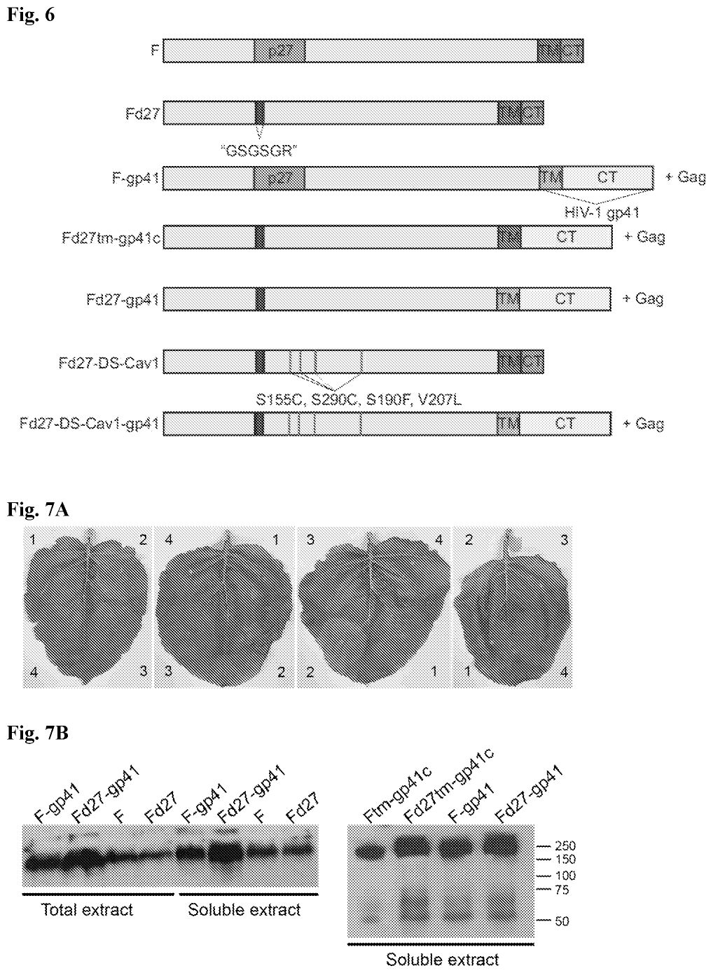

Like HIV-1 glycoprotein gp41, RSV F is a type I fusion protein presented in trimers embedded in a viral membrane. Various RSV-F variants and their fusions with truncated HIV-1 gp41 were designed and constructed (FIG. 6).

The detailed atomic resolution and structural studies elucidated RSV pre-F structure and its potent antigenicity have created interest in stabilizing pre-F conformation as a potential component of an RSV vaccine. This led to the design of pre-F stabilized subunit vaccine DS-Cav1 that recently showed promising results in phase I clinical trial. In contrast to RSV F vaccine candidates that boost mainly F binding antibodies, pre-F stabilized immunogen induces site Ø-specific antibodies that correlate with neutralizing antibody activity. Moreover, the evidence suggests that these highly potent neutralizing sites on pre-F are available when they are presented in trimers, hence, various trimerization promoting domains, particularly bacteriophage T4 fibritin protein (foldon), are incorporated in the antigen design.

In an effort to deliver pre-F stabilized antigen mimicking native structure, several constructs were designed by engineering F to include previously identified pre-F stabilizing mutations and fusing it into our established HIV-1 VLP system (Meador et al., “A heterologous prime-boosting strategy with replicating Vaccinia virus vectors and plant-produced HIV-1 Gag/dgp41 virus-like particles.” Virology, 2017, 507: 242-256) for the presentation of chimeric trimerized pre-F in enveloped VLPs. To stabilize F in pre-F, the furin-cleavage fragment p27 was removed from native F protein and replaced it with short linker GSGSGR (SEQ ID NO. 1) to create Fd27 variant as its inclusion showed to destabilize trimer formation and pre-F conformation. Further modifications included fusing both F and Fd27 variants to truncated gp41, which initially incorporated F TM fusion to gp41 cytosolic domain to produce Ftm-gp41c or Fd27tm-gp41c. Then F TM was substituted with gp41 TM to make F-gp41 and Fd27-gp41. All of these constructs were tested for expression in plants in multiple leaves (FIG. 7A). The constructs with gp41 were also tested for co-expression with Gag, which did not significantly alter their expression profile. Since, Fd27 variant initially showed higher expression, subsequent pre-F stabilizing mutations (DS-Cav1) were added to that construct and tested their expression in plants as well.

a. F, Fd27 and gp41 Fusion Constructs

Plasmids harboring plant-optimized RSV F fused to HIV-1 VLPs were constructed as follows. The pVG100 plasmid containing F gene from RSV A2 strain (FruitVaccine, Inc.) was used to get native F gene (pVG100) and also to make Fd27 version (pVG100-d27) which contained deletion of p27 and replacing the linker with “GSGSGR” (SEQ ID NO. 1).

F gene was PCR amplified with primers Q26-Bsa-F and I526-Bsa-R to get the ectodomain only. Gene fragment was then digested with SpeI-BsaI. Dgp41 was PCR amplified with primers gpTM-Bsa-F and Ext3-R to get the transmembrane (TM) and cytosolic domains (C), then digested with BsaI-SacI. Backbone (pBYKEAM-BARSV-F) was digested with SpeI-SacI and the fragments were ligated to produce geminiviral replicon vector construct pBYKEAM-BAF-gp41.

1. In accordance with certain embodiments, an outline of steps for constructing pBYKEAM-BAF-gp41 (F ectodomain fused to gp41 TM and cytosolic domain)
Backbone: pBYKEAM-BARSV-F (3/17/20) digested with SpeI-SacI
Insert #1: PCR product of pVG100 (11/20/19) with primers Q26-Bsa-F and I526-Bsa-R digested with Spe-BsaI (1427 bp)
Insert #2: PCR product of pBYKEAM-dgp41 (4/25/18) with primers gpTM-Bsa-F and Ext3-R digested with BsaI-SacI (566 bp)
Digested products from these 3 above: backbone and 2 inserts were ligated, transformed into E. coli DH5a cells.
Colonies were screened using primers S215P-Bsa-F and Ext3-R. The bands were compared with pBYKEAM-BARSV-F plasmid (1178 bp) to the expected band of 1596 bp.
Positive colonies selected and used to grow overnight cultures. They were used for plasmid isolation, diagnostic digestion (XhoI-SacI) and confirmation with sequencing (primers used Q26-Bsa-F, S215P-Bsa-F, and Ext3-R). The bacterial stock with the confirmed plasmid was then saved in −80° C. as a glycerol stock.
Confirmed plasmid was also used to transform into Agrobacterium tumefaciens EHA105 cells and confirmed with the PCR colony screen using the same primers before use in protein expression.

F gene was PCR amplified with primers Q26-Bsa-F and C550-Bsa-R to get the ectodomain and TM. Gene fragment was then digested with SpeI-BsaI. Dgp41 was PCR amplified with gpC-Bsa-F and Ext3-R to get the C domain only, then digested with BsaI-SacI. These two gene fragments were then ligated with SpeI-SacI digested backbone pBYEKAM-BARSV-F to get geminiviral replicon vector construct pBYKEAM-BAFtm-gp41c.

2. In accordance with certain embodiments, an outline of steps for constructing pBYKEAM-BAFtm-gp41 (F ecto+TM fused to gp41 cytosolic domain)
Backbone: pBYKEAM-BARSV-F (3/17/20) digested with SpeI-SacI
Insert #1: PCR product of pVG100 (11/20/19) with primers Q26-Bsa-F and C550-Bsa-R digested with Spe-BsaI (1492 bp)
Insert #2: PCR product of pBYKEAM-dgp41 (4/25/18) with primers gpC-Bsa-F and Ext3-R digested with BsaI-SacI (492 bp)
Digested products from these 3 above: backbone and 2 inserts were ligated, transformed into DH5a cells.
Colonies were screened using primers S215P-Bsa-F and Ext3-R. The bands were compared with pBYKEAM-BARSV-F plasmid (1178 bp) to the expected band of 1587 bp.
Positive colonies selected and used to grow overnight cultures. They were used for plasmid isolation, diagnostic digestion (XhoI-SacI) and confirmation with sequencing (primers used Q26-Bsa-F, S215P-Bsa-F, and Ext3-R). The bacterial stock with the confirmed plasmid was then saved in −80 C as a glycerol stock.
Confirmed plasmid was also used to transform into Agrobacterium tumefaciens EHA105 cells and confirmed with the PCR colony screen using the same primers before use in protein expression.

Similar genetic cloning was performed to get pBYKEAM-BAFd27-gp41 and pBYKEAM-BAFd27tm-gp41c using pVG100-d27 instead of pVG100, resulting in similar modification made to Fd27 instead of F.

3. In accordance with certain embodiments, an outline of steps for constructing pBYKEAM-BAFd27-gp41 (Fd27 is modified to delete the p27 peptide and replace it with a linker “GSGSGRSLG” (SEQ ID NO. 4))
Backbone: pBYKEAM-BARSV-F (3/17/20) digested with SpeI-SacI (13,547 bp)
Insert #1: PCR product of pVG100-d27 (1/24/20) with primers Q26-Bsa-F and I526-Bsa-R digested with Spe-BsaI (1355 bp)
Insert #2: PCR product of pBYKEAM-dgp41 (4/25/18) with primers gpTM-Bsa-F and Ext3-R digested with BsaI-SacI (566 bp)
Digested products from these 3 above: backbone and 2 inserts were ligated, transformed into DH5a cells.
Colonies were screened using primers S215P-Bsa-F and Ext3-R. The bands were compared with pBYKEAM-BARSV-F plasmid (1178 bp) to the expected band of 1596 bp.
Positive colonies selected and used to grow overnight cultures. They were used for plasmid isolation, diagnostic digestion (XhoI-SacI) and confirmation with sequencing (primers used Q26-Bsa-F, S215P-Bsa-F, and Ext3-R). The bacterial stock with the confirmed plasmid was then saved in −80 C as a glycerol stock.
Confirmed plasmid was also used to transform into Agrobacterium tumefaciens EHA105 cells and confirmed with the PCR colony screen using the same primers before use in protein expression.

4. In accordance with certain embodiments, an outline of steps for constructing pBYKEAM-BAFd27tm-gp41 (Fd27 is modified to delete the p27 peptide and replace it with a linker “GSGSGRSLG” (SEQ ID NO. 4))
Backbone: pBYKEAM-BARSV-F (3/17/20) digested with SpeI-SacI
Insert #1: PCR product of pVG100-d27 (1/24/20) with primers Q26-Bsa-F and C550-Bsa-R digested with Spe-BsaI (1492 bp)
Insert #2: PCR product of pBYKEAM-dgp41 (4/25/18) with primers gpC-Bsa-F and Ext3-R digested with BsaI-SacI (492 bp)
Digested products from these 3 above: backbone and 2 inserts were ligated, transformed into DH5a cells.
Colonies were screened using primers S215P-Bsa-F and Ext3-R. The bands were compared with pBYKEAM-BARSV-F plasmid (1178 bp) to the expected band of 1587 bp.
Positive colonies selected and used to grow overnight cultures. They were used for plasmid isolation, diagnostic digestion (XhoI-SacI) and confirmation with sequencing (primers used Q26-Bsa-F, S215P-Bsa-F, and Ext3-R). The bacterial stock with the confirmed plasmid was then saved in −80 C as a glycerol stock.
Confirmed plasmid was also used to transform into Agrobacterium tumefaciens EHA105 cells and confirmed with the PCR colony screen using the same primers before use in protein expression.

b. DS-Cav1 Mutants

Pre-fusion stabilizing mutations to Fd27 were added to introduce further pre-F stabilizing DS-Cav1 mutations (disulfide bond and cavity filling). The 4 mutations (S155C, S290C, S190F, V207L) were done separately and sequentially. For backbone, pBYKEAM-BARSV-F was digested with SpeI-SacI. For inserts, pVG100-d27 was PCR amplified with Q26-Bsa-F and S155C-Bsa-R and digested with SpeI-BsaI to get 248 bp fragment; pVG100-d27 was PCR amplified with S155C-Bsa-F and NOS, then digested with BsaI-SacI to get 1274 bp fragment. These 3 fragments were combined with a ligase to get pBYKEAM-BAFd27-155C construct. Diagnostic restriction digestion was performed and was further confirmed with Sanger sequencing before proceeding to the next step.

To add the second mutation (S290C), pBYKEAM-BAFd27-155C was used as a backbone and digested with SpeI-SacI. For inserts, the same plasmid was PCR amplified with Q26-Bsa-F and S290C-Bsa-R, then digested with SpeI-BsaI to get 706 bp fragment; also same plasmid was PCR amplified with S290C-Bsa-F and Ext3-R, then digested with BsaI-SacI to get 878 bp fragment. Colonies were screened with PCR and confirmed with Sanger sequencing to confirm this new construct pBYKEAM-BAFd27-DS.

To add the third mutation, pBYKEAM-BAFd27-DS was cut with SpeI-SacI to separate the backbone. To make the inserts, pBYKEAM-BAFd27-DS was PCR amplified with Q26-Bsa-F and S190F-Bsa-R, then digested with SpeI-BsaI to get 340 bp fragment, it was also amplified with S190F-Bsa-F and Ext3-R, then digested with BsaI-SacI to get the 1169 bp fragment. All fragments were ligated to produce pBYKEAM-BAFd27-DS-190F construct.

Finally, to introduce the last mutation of DS-Cav1, pBYKEAM-BAFd27-DS-190F plasmid was digested with SpeI-SacI. For inserts, it was PCR amplified with Q26-Bsa-F and V207L-Bsa-R, then digested with SpeI-BsaI to get 405 bp fragment; it was also amplified with V207L-Bsa-F and Ext3-R, then digested with BsaI-SacI to get 1117 bp gene fragment. All 3 fragments were ligated to produce final pBYKEAM-BAFd27-DS-Cav1 plasmid.

Optimized construction of p55 Gag (subtype C R5 HIV-1 isolate, 1084i, GenBank Accession no. AY805330) has been previously described.

1. In accordance with certain embodiments, an outline of steps for introducing S155C to make pBYKEAM-BAFd27-155C
Backbone: pBYKEAM-BARSV-F (3-12-20) digested with SpeI-SacI (13547 bp)
Insert #1: PCR product of pVG100-d27 (1-24-20) with primers Q26-Bsa-F and S155C-Bsa-R (347 bp) digested with SpeI-BsaI (248 bp)
Insert #2: PCR product of pVG100-d27 (1-24-20) with primers S155C-Bsa-F and NOS (1346 bp) digested with BsaI-SacI (1274 bp)
Digested products from these 3 above: backbone and 2 inserts were ligated, transformed into E. coli DH5a cells.
Colonies were screened using primers Q26-Bsa-F and S155C-Bsa-R (347 bp) and compared to pBYKEAM-BARSV-F (413 bp)
Prep plasmid, digest SpeI-Xho (465, 235 bp) vs. pBYKEAM-BARSV-F (531, 235 bp).
Two PCR positive colonies were grown overnight and plasmids were isolated. Diagnostic digestion was performed using SpeI and XhoI (expected fragments 465 bp and 235 bp compared to pBYKEAM-BARSV-F (531 bp and 235 bp). The bacterial stock with the confirmed plasmid was then saved in −80 C as a glycerol stock.
One digest-confirmed plasmid was sequenced with primer G51-Bam-F to confirm S155C mutation (AGC->TGC) and PCR-amplified sequence with Q26-Bsa-F and Ext3-R.

2. In accordance with certain embodiments, an outline of steps for constructing pBYKEAM-BAFd27-DS
Backbone pBYKEAM-BAFd27-155C digested with SpeI-SacI
Insert #1: PCR product of pBYKEAM-BAFd27-155C with primers Q26-Bsa-F and S290C-Bsa-R (818 bp)] digested with SpeI-BsaI (706 bp)
Insert #2: PCR product of pBYKEAM-BAFd27-155C with primers S155C-Bsa-F and Ext3-R (951 bp) digested with BsaI-SacI (878 bp)
Digested products from these 3 above: backbone and 2 inserts were ligated, transformed into E. coli DH5a cells.
Colonies were screened with primers Q26-Bsa-F and S290C-Bsa-R, 53° (818 bp) vs. pBYKEAM-BAFd27-155C (818 bp)
Prep plasmids 2 PCR positives; digest SpeI-SacI, confirm 1509 bp fragment, make glycerol cell stocks.

c. Primers Used in the Construction of the Plasmids

Table 1 lists the primers used along with their sequences.

TABLE 1

| | | SEQ ID |
|---|---|---|
| Name | Sequence | NO. |
| Q26-Bsa-F | aggGGTCTCgTGGTCAAAACATAACCGAGGAGTTC | 4 |
| I526-Bsa-R | ggGGTCTCatAATGTTAGTGGTGGACTTGC | 5 |
| gpTM-Bsa-F | ggGGTCTCcATTaagattttcattatgattgtggg | 6 |
| Ext3-R | cttcttcttcttcttttctcattgtc | 7 |
| C550-Bsa-R | ggGGTCTCgttACAATACAAAAGTAAACCCACTG | 8 |
| gpC-Bsa-F | ggGGTCTCgGTaacagagttaggcaaggatac | 9 |
| S155C-Bsa-R | gtGGTCTCTGcaaACAGCGACACCAGAGGC | 10 |
| S155C-Bsa-F | gtGGTCTCAttgCAAAGTTCTCCATTTGGAAGG | 11 |
| NOS | CGGCAACAGGATTCAATC | 12 |
| S290C-Bsa-R | gtGGTCTCTAcACATTATGCTATAGCTTTGCTGAC | 13 |
| S290C-Bsa-F | gtGGTCTCTGTgTATCATCAAAGAAGAAGTGCTTGC | 14 |
| S190F-Bsa-R | gtGGTCTCTgaAGGTCAAAACTGAAACTCCATTA | 15 |
| S190F-Bsa-F | gtGGTCTCCCTtcAAGGTCTTGGACCTTAAGAAC | 16 |
| V207L-Bsa-R | gtGGTCTCTTGAgTATAGGCAATAACTGTTTGTCA | 17 |
| V207L-Bsa-F | gtGGTCTCAcTCAAcAAGCAATCTTGTAGTATTAGCAAC | 18 |

### II. Agroinfiltration and Protein Isolation Methods

All constructs were transformed into A. tumefaciens strain EHA105 by electroporation. LB agar plates with the transformants were grown for 2 days in 30° C. Grown colonies were confirmed with PCR colony screen and grown overnight shaking at 30° C. Cultures were centrifuged at 4,000×g for 20 min at room temperature, supernatant was removed and the pellets were resuspended in infiltration buffer (10 mM 2-(N-morpholino) ethanesulfonic acid, 10 mM magnesium sulfate, pH 5.5) to get OD600=0.4. For co-expression of two separate constructs for VLP assembly, same volumes of each constructs were pre-mixed. Infiltration proceeded by taking the leaves of 5-6 weeks old glycol-engineered N. benthamiama (GnGn) and slightly puncturing to inject the bacterial suspensions with needless syringes either to get spot infiltrations (small-scale) or whole leaf infiltration (large-scale). Infiltrated plant leaves were harvested at 4 days post-infiltration (DPI).

a. Small Scale Extraction

To extract F proteins for small scale analysis, 200 mg of infiltrated leaf spots were excised and placed in 2 mL tubes with 1 mL of cold extraction buffer (25 mM sodium phosphate, 100 mM NaCl, 1 mM EDTA, 50 mM sodium ascorbate, 2 mM PMSF, pH 7.8). This was homogenized in Bullet Blender machine with metal beads inside the tubes. The extract was then centrifuged at 14,000×g for 20 min, 4° C. and supernatant was analyzed with western blot.

b. Large Scale Extraction and Enrichment

Transiently expressed VLP forming F constructs were extracted as follows. Harvested plant material was homogenized in 1:3 (w:v) cold extraction buffer (25 mM sodium phosphate, 100 mM NaCl, 1 mM EDTA, 50 mM sodium ascorbate, 2 mM PMSF, pH 7.8). To enhance protein solubilization, the extract was stirred at 4° C. for 30 min. After, the extract was strained through miracloth, it was centrifuged at 14,000×g for 20 min, 4° C. and supernatant was subjected to 10% (only for DS-Cav1-gp41 with Gag) or 20% (the rest of constructs) ammonium sulfate to precipitate VLPs and eliminate major plant proteins. Supernatant with ammonium sulfate was stirred for 1 hr, then centrifuged at 36,000×g, 30 min, 4° C. The collected pellet was resuspended in minimal 1×PBS buffer (140 mM NaCl, 2 mM KCl, 10 mM Na₂HPO₄, 1 mM KH₂PO₄, pH 7.4), briefly centrifuged. For VLP enrichment, the sample was subjected to iodixanol density cushion ultracentrifugation (10%, 20%, 50% Optiprep). The 20% Optiprep fraction was sterilized using 0.2 μm syringe filters (Titan3, Thermo Fisher Scientific) and loaded on centrifugal devices with 300 MWCO membrane (Sartorius) to concentrate and iodixanol removal with buffer exchange at 4,000×g, 4° C. Quantitative Western blot was used to measure F yield by using serially diluted commercial F protein (Sino Biological).

### III. Methods Related to Comparing Protein Expression

a. Antibody Construction and Expression

Variable regions of CR9501 and CR9503 antibodies were constructed into humanized 6D8 IgG. CR9501 antibody binds only to pre-F conformation and have variable heavy (VH) and light chain (VL) regions from 58C5. To make this construct, intermediate vector pUC-6D8H!S and plant-optimized CR9501 VL gene (gblock 6OE4_B) were digested with XhoI-NheI and self-ligated to make pUC-CR9501G1. After the gene was confirmed with Sanger sequencing. Next, expression vector pBY11HA-GFP and plant-optimized CR9501 VH gene (gblock 6OE4_C) were cut with XbaI-SacI and self-ligated to produce pBY11HA-CR9501K. This was confirmed with Sanger sequencing. To make the final double expression cassette that includes both CR9501 VH and VL, pBY11HA-CR9501K was used as a backbone and cut with SbfI-XbaI, pUC-CR9501G1 was cut with SbfI-BsrGI to use 1641 bp fragment, pBY11HA-h6D8 was digested with BsrGI-XbaI to use 3273 bp constant region for heavy chain. These were used for 3 fragment ligation to produce pBY11HA-CR9501.

CR9503 antibody binds both pre-F and post-F conformations. It contains VH and VL regions from motavizumab. For this construct, expression vectors were separate for heavy (HC) and light chains (LC) that could be co-expressed by combining them before plant infiltration. To make CR9503 HC, backbone pUC-CR9501G1 and plant-optimized CR9503 HC gene (gblock Mota-HC) were cut with XhoI-NheI and self-ligated to produce intermediate pUC-CR9503H plasmid. After confirming the gene with Sanger sequencing, pUC-CR9503H and pBYKEAM-BAGFPas6H were then cut with XbaI-SacI and self-ligated to make final CR9503 HC expression vector—pBYKEAM-CR9503H. To make CR9503 LC expression vector, pBYKEAM-BAGFPas6H and plant-optimized CR9503 LC gene (gblock Mota-LC) were digested with XbaI-SacI and after sequence confirmation self-ligated to produce pBYKEAM-CR9503L.

For transient expression in plants, they were all transformed into Agrobacterium EHA105 strain and infiltrated plants as for F proteins. CR9501 was expressed alone, as it has double cassette for both heavy and light chains. While CR9503 was infiltrated by mixing heavy and light chain in equal OD600 for co-expression.

b. Antibody Purification

Harvested plants were extracted as above, homogenized in 1:3 (w:v) cold extraction buffer (25 mM sodium phosphate, 100 mM NaCl, 1 mM EDTA, 50 mM sodium ascorbate, 2 mM PMSF, pH 7.8), then the extract was stirred at 4° C. for 30 min and strained through miracloth. It was then centrifuged at 14,000×g for 20 min, 4° C. and supernatant was filtered using 0.75 μm glass fiber filters (Titan3, Thermo Fisher Scientific) and antibodies are purified from the extract using Protein G chromatography as described in detail (Kamzina et al. 2021). Purified antibody concentration was determined by optical absorbance at 280 nm and quality was analyzed with SDS-PAGE and western blot.

c. SDS-PAGE and Western Blot

Gag and F were analyzed in extractions by mixing the sample with SDS sample buffer (50 mM Tris-HCl, pH 6.8, 2% SDS, 10% glycerol, 0.02% bromophenol blue) and resolving in 4-15% stain-free polyacrylamide gels (Bio-Rad). Samples in reducing conditions were used with 0.1 M DTT and boiled for 5 min. The stain-free gels were first visualized under UV then transferred to nitrocellulose membrane (Bio-Rad) for immunoblotting.

To detect proteins, the membrane was first blocked in PBST (1×PBS, 0.05% Tween) with 5% dry milk at RT for 30 min, rotating. For Gag detection, the membrane was probed with anti-HIV-1 p24 Gag monoclonal antibody (NIH-ARP Cat #6458), followed by HRP conjugated anti-mouse IgG (CalBiochem); to detect F, it was probed with either anti-F (Sino Biological) followed by anti-mouse IgG-HRP (CalBiochem). Quantitative western blotting was used to quantify Gag and F by using p24-CTA2 (Kessans et al. 2013) and RSV-F (A2) (Sino Biological), respectively.

d. Dot Blot

For dot blot, 2 μL of plant extracts or 56 ng of RSV F antigen standard (Sino Biological) were spotted on nitrocellulose membranes and dried for 3 min. The membranes were blocked with PBST containing 5% dry milk at room temperature. Then, they were probed with 1 μg/mL of either CR9503 or CR9501 for detection of total F or F in prefusion conformation, respectively, followed by anti-human IgG-HRP (Santa Cruz).

e. Mouse Immunizations

All animals were handled in accordance to the Animal Welfare Act and Institutional Animal Care and Use Committee (IACUC), Arizona State University.

Female 6-8 weeks old BALB/c mice, 6 per group, were used for immunization studies. For SC administration, each mouse received F constructs (5 μg Fd27, 1 μg DS-Cav1) or F VLP fusions (5 μg of Fd27-gp41 with Gag, 1 μg of Fd27-DS-Cav1-gp41 with Gag) mixed with Imject Alum (Thermo Scientific) in 1:3 raio of Alum to immunogen were used as well as PBS control. For IN delivery, 1 μg of Fd27-gp41 with Gag mixed with 11.1 ng of Cholera Toxin (List Labs) was delivered in 10 μL with a pipette into each nostril. All experimental vaccines were delivered in 3 doses with 3 week intervals. Serum was collected before every immunization and 3 weeks after the last dose, while nasal flush and fecal samples were collected after mice were euthanized.

f. IgG and IgA Measurement with ELISA

Mouse antibody titers in serum for IgG and mucosal sites (nasal flush and fecal samples) for IgA were measured with ELISA. To quantify IgG and IgA against, 96-well high binding polystyrene plates (Corning) were coated with 0.25 μg/mL of F (Sino Biological) overnight at 4° C. to detect anti-F antibodies. After blocking with 5% non-fat dry milk in PBST for 1 hr at RT, threefold serially diluted mouse samples were added to the wells starting at 1:50 (serum), or twofold serially diluted mucosal samples were added starting at 1:2 (fecal) and 1:5 (nasal flush) and incubated 1 hr at 37° C. Bound IgG and IgA antibodies were detected with HRP-conjugated anti-mouse IgG (CalBiochem) or IgA (Sigma), incubating 1 hr at 37° C. Plates were washed with PBST after each step, developed with TMB substrate (Thermo Fisher Scientific) and the reaction was stopped with 1 M HCl. The absorbance was read at 450 nm. Endpoint titers were Endpoint titers (EPT) were taken as the reciprocal of the lowest dilution which produced an OD450 signal twice the background.

g. Statistics

All statistical analyses were done using GraphPad Prism Software. To determine statistical significance one-tailed ANOVA followed by the Kruskal-Wallis test was used. Significance cut-off was defined as p<0.05.

### IV. Expression of Chimeric F VLPs

Multiple expression studies were performed to test different leaves and different plants to eliminate particular expression influences.

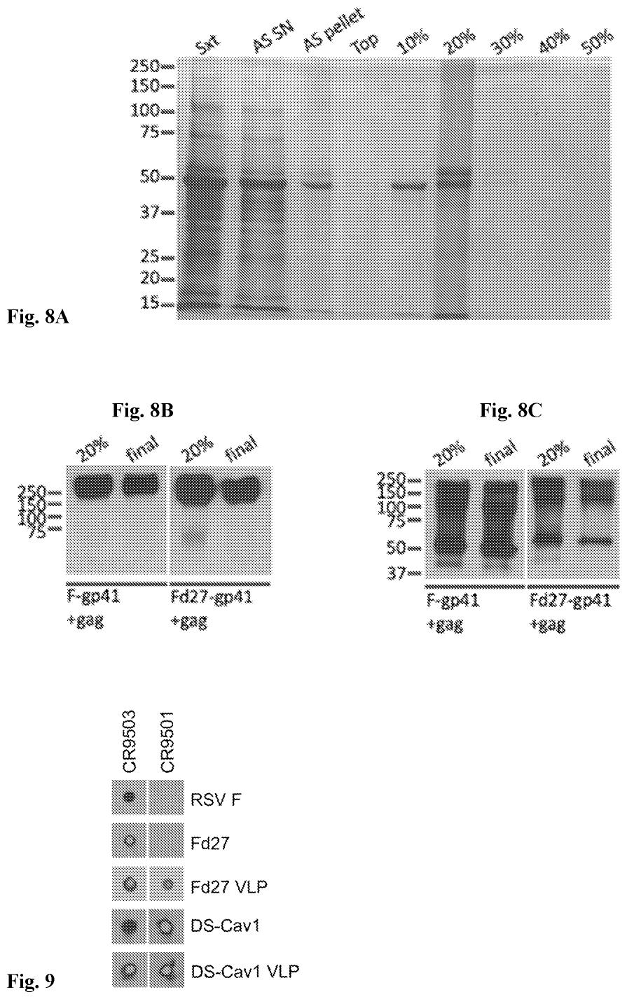

Wp27 deletion resulted in higher expression on Western Blot (FIGS. 7B, 8B, and 8C). This can also be confirmed in spot infiltration with visible necrosis, which usually corresponds to higher expression (FIG. 7A). 1.5 to 3-fold higher yield was consistently observed with Fd27-gp41 compared to other constructs, hence, subsequent studies utilized Fd27-gp41 presumably assembled into VLPs when co-expressed with Gag (Fd-27 VLPs). Moreover, although it was not directly compared and quantified, incorporating gp41 TM instead of F TM seemed to enhance the expression of these VLPs from western blot analysis (data not shown). On the other hand, DS-Cav1 construct had the lowest yield compared to the rest, hence, a reduced amount (1 μg) was used for mouse immunization and an equivalent amount was used for DS-Cav1 VLPs for comparison. The DS-Cav1 construct could be further modified to include his-tags for easier purification and increased yield.

### V. Confirming Pre-F Conformation

To differentiate between pre-F and post-F conformations, pre-F specific CR9501 (variable region adapted from D25 antibody) and conformation independent CR9503 (variable region from motavizumab) F recognizing antibodies were constructed and produced in plants. Since CR9501 recognizes pre-F distinctive antigenic site Ø located on the membrane-distal apex of pre-F trimers, it is convenient to use it for detecting pre-F conformation. On the other hand, CR9503 antibody that recognizes site II present in both pre-F and post-F was used to detect total F.

A dot blot with CR9501 and CR9503 antibodies was used for a brief analysis of pre-F conformation of the described constructs (FIG. 9). This revealed that Fd27 VLPs and DS-Cav1 VLPs were mostly in pre-F conformation, and, as expected, commercial RSV F protein was not recognized by CR9501 but was recognized by conformation independent antibodies CR9503. On the other hand, DS-Cav1 and Fd27 that are expected to be monomers are either mostly or all in post-F, respectively (FIG. 9).

### VI. F-Specific IgG Titers in Mouse Serum

VLP-based vaccines are a well-established strategy that have an advantageous profile over other types of vaccines. It has been reported that stimulation of both cellular and humoral immune responses were observed with a HIV-1 VLP platform (Meador et al. 2017), hence whether the chimeric version presenting pre-F would similarly induce these responses was tested.

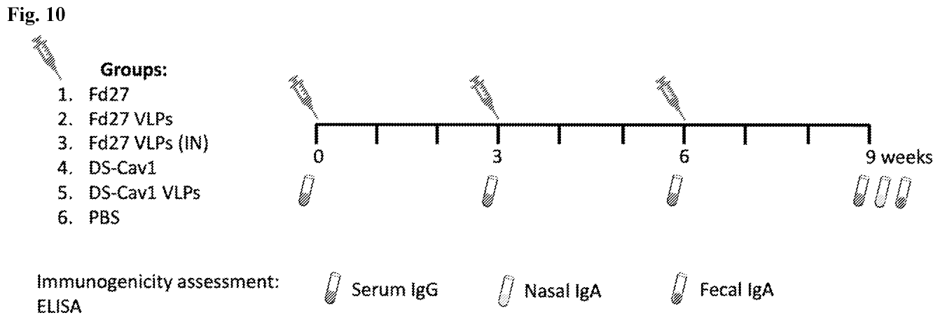

The described vaccine platform was well tolerated and immunogenic in mice. For immunogenicity studies, BALB/c mice divided into 6 groups were immunized SC either with PBS, 5 μg of Fd27 or Fd27 VLPs, 1 μg of DS-Cav1, or DS-Cav1 VLPs, or were administered IN 1 μg of Fd27 VLPs (FIG. 10). DS-Cav1 construct had the lowest yield compared to the rest, hence, 1 μg was used for SC immunization and used an equivalent amount for DS-Cav1 VLPs. For immune response evaluation, serum samples were collected before every dose and 3 weeks after the final injection (FIG. 10). IgG and IgA in treated mice were measured for humoral response analysis; and the relative levels of IgG subtypes to use as an approximation to define bias in the cellular response towards Th1 or Th2 cells.

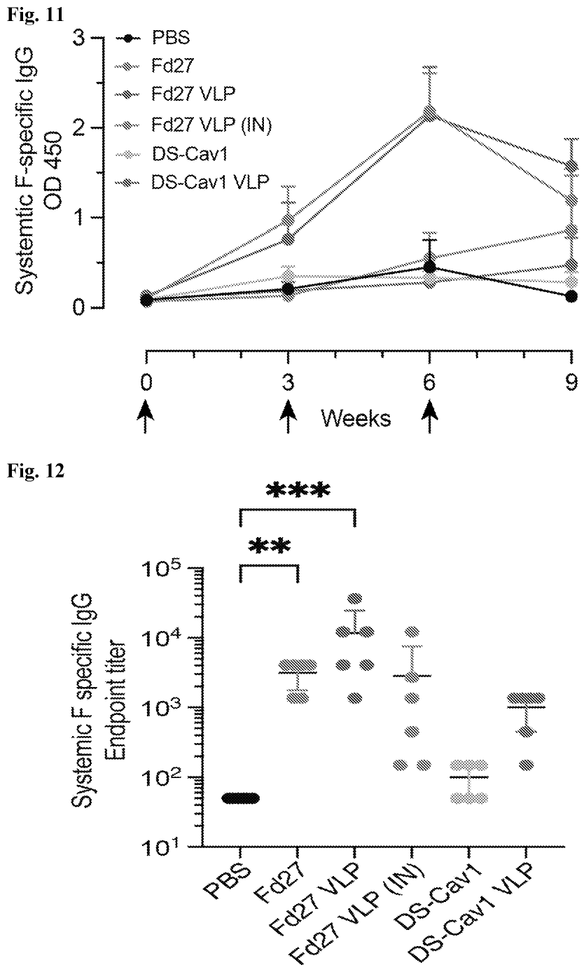

FIG. 11 displays systemic anti-F IgG titers that was collected over the course of the experiment. Interestingly, IgG titers for both Fd27 groups started steeply increasing immediately after the first dose. However, EPTs showed a significantly high IgG response for group that received Fd27 (p=0.0038) and even higher for Fd27 VLPs (p=0.0002) compared to PBS control (FIG. 12). Fd27 VLPs exhibited more than 8-fold higher EPTs compared to its non-VLP version. Similarly, DS-Cav1 VLP group showed higher IgG EPTs than DS-Cav1, but both showed low titers, which could be due to receiving 5 times less antigen than Fd27 groups.

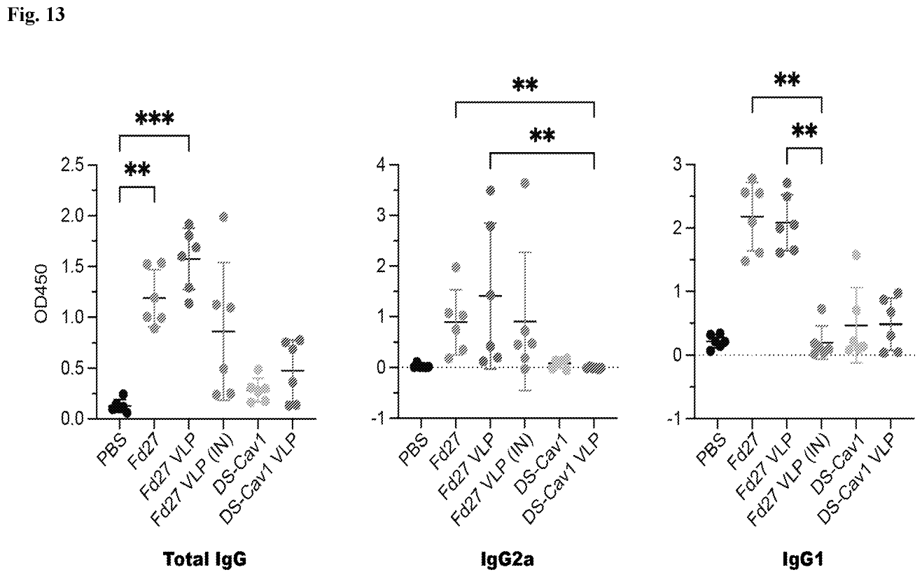

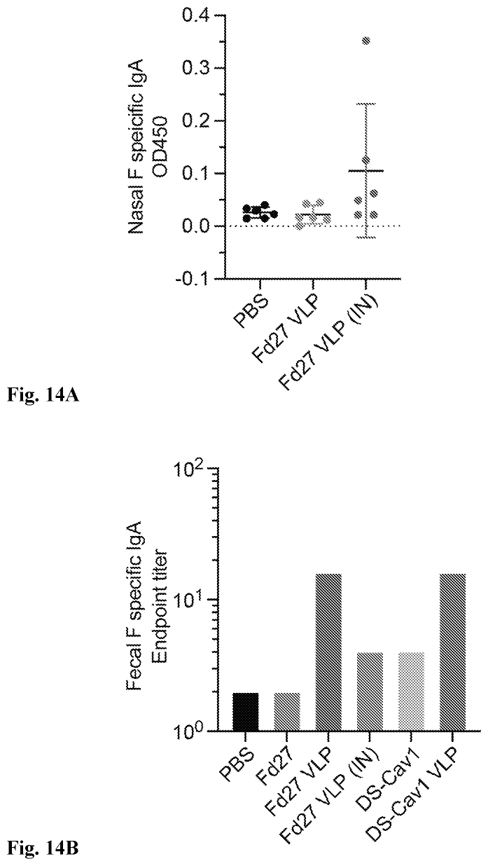

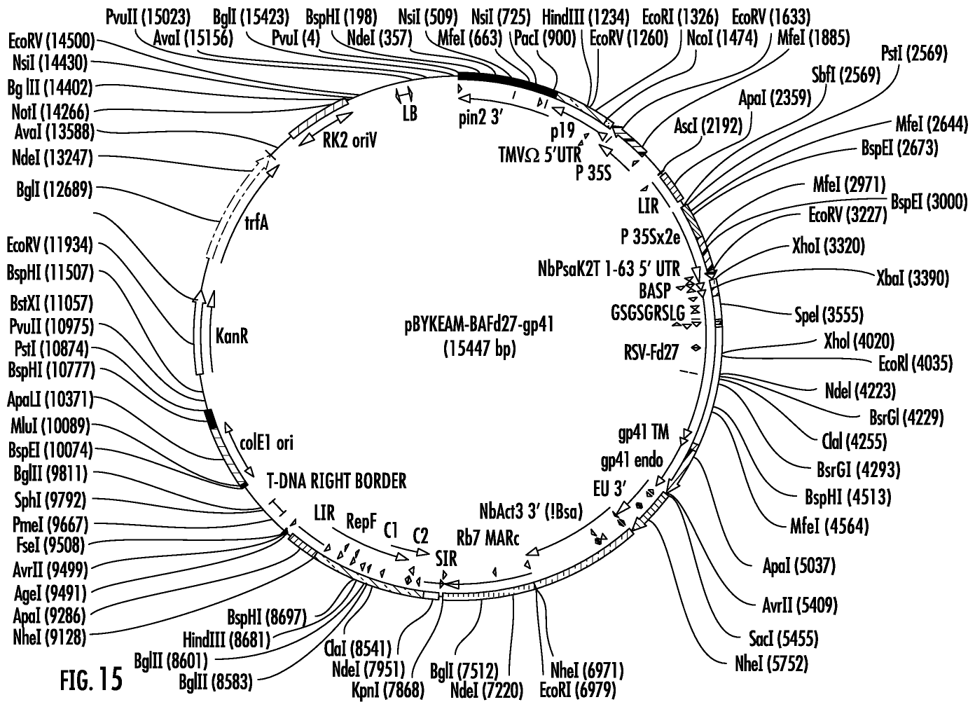

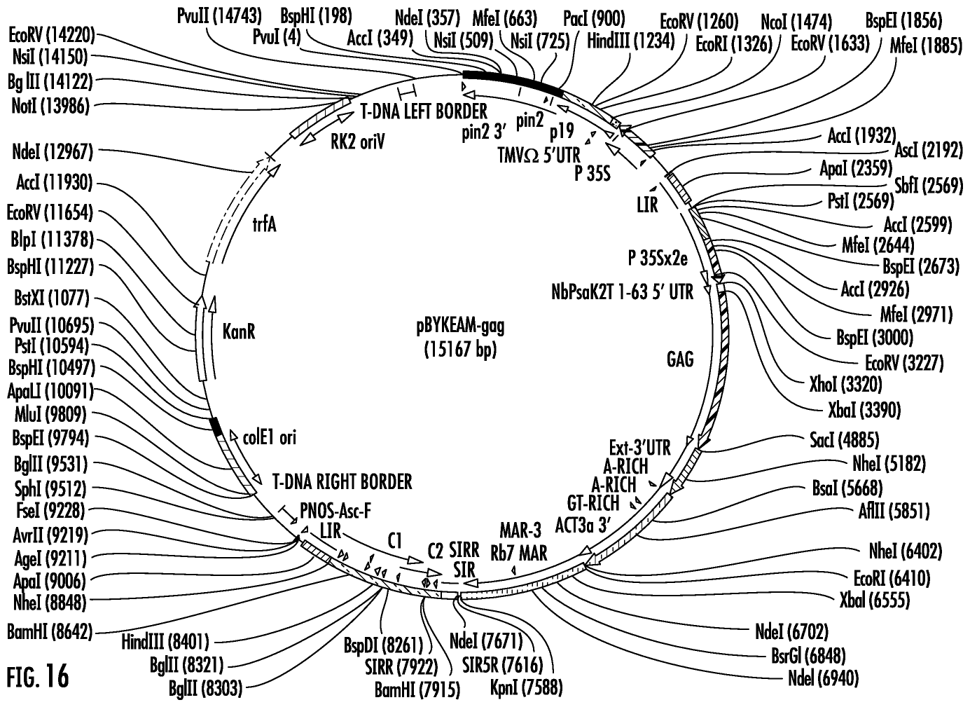

IgG subtypes were used as markers to distinguish between T helper cell (Th) 1 and Th2 immune responses. Due to the absence of a standard curve for IgG2a and IgG1, only groups with their VLP versions from each graph could be compared (FIG. 13). Obtained results from the ratio of mean values suggest that Fd27 VLP has a higher titer (IgG2a/IgG1=0.7) than Fd27 alone (0.4). However, among all groups, intranasal administration had the highest ratio (4.5), implying Th1-skewed immune response.

While total IgG levels were significantly high for both Fd27 and Fd27 VLP groups compared with the control group, the latter showed higher response consistent with recognized VLP benefits. On the other hand, IgG2a/IgG1 ratio gave a high number for IN delivered Fd27 VLP, suggesting Th1-skewed immune response, though further studies are needed before the induced immune responses could conclusively be described as being Th1-skewed.

### VII. Mucosal Antibody Titers

Since RSV infection is a respiratory disease, it is essential to induce immunity in the mucosal sites. Moreover, studies have shown that control in the upper respiratory is mainly associated with anti-viral IgA titers, boosting neutralizing antibodies and complement activation thus, generating protective immunity in this site is essential to consider when developing vaccine strategies. The vaccine platform for generating an immune response in mucosal sites, Fd27 VLPs were delivered intranasally according to the schedule shown in FIG. 10.

To measure F-specific nasal IgA titers, nasal flush from groups that received SC Fd27 VLP, IN Fd27 VLP and PBS were collected. Although subcutaneously administered vaccine received 5 times more of the antigen (5 μg compared with 1 μg), intranasally administered vaccine exhibited much higher nasal IgG titers compared with SC delivery (p>0.09).

To measure fecal anti-F IgA titers, pooled fecal pellets were collected from group cages at the end of the experiment. Interestingly, both VLP groups demonstrated increased IgA titers compared to non-VLP versions, consistent with known VLP ability to stimulate an advantageous immune response.

The immunogenicity findings suggest that intranasal administration indeed induced higher IgA levels (p=0.09) compared to subcutaneous administration of the same immunogen in a higher dose (5 μg vs 1 μg). This did not reach significance, presumably due to the wide range of data distribution, which could be attributable to the difficulties of nasal administration of antigens delivered in small volume (10 μL/per nostril). Due to challenges associated with intranasal delivery conventionally, intranasally administered immunogens contain a higher dose of antigen compared to parenteral administration. However, the results obtained here are promising since a slightly higher IgA response in nasal flush indicates that antigens reached the mucosal sites, crossed the mucosal layer, and stimulated local IgA generation. Further improvement could be achieved by increasing the amount of antigens.

We claim:

1. A human immunodeficiency virus (HIV) virus-like particle (VLP) comprising:
a Gag protein;
a fragment of gp41 protein comprising the C-terminal cytosolic domain of the gp41 protein; and
an antigen,
wherein the antigen is not a peptide or protein from HIV and is linked to the fragment of gp41 protein.

2. The HIV VLP of claim 1, wherein the antigen is displayed on the VLP surface.

3. The HIV VLP of claim 1, wherein the antigen is trimerized.

4. The HIV VLP of claim 1, wherein the fragment of gp41 protein further comprises the transmembrane domain of the gp41 protein.

5. The HIV VLP of claim 4, wherein the fragment of gp41 protein consists of the transmembrane domain and the cytosolic domain of the gp41 protein.

6. The HIV VLP of claim 4, wherein the fragment of gp41 protein further comprises the membrane-proximal external region (MPER) of the gp41.

7. The HIV VLP of claim 4, wherein the C-terminus ectodomain of the antigen is linked to the N-terminus of the transmembrane domain of the gp41 protein.

8. The HIV VLP of claim 4, wherein the C-terminus of the transmembrane domain of the antigen is linked to the N-terminus of the cytosolic domain of the gp41 protein.

9. The HIV VLP of claim 1, wherein the antigen is a fragment of a protein selected from the group comprising: Influenza HA protein, SARS-CoV Spike (S) protein, SARS-CoV-2 S protein, and respiratory syncytial virus (RSV) F protein.

10. The HIV VLP of claim 9, wherein the antigen is a fragment of RSV F protein, the fragment of RSV F protein lacks the p27 region.

11. The HIV VLP of claim 10, wherein the fragment of the RSV F protein further lacks the cytosolic domain and the transmembrane domain of the RSV F protein.

12. The HIV VLP of claim 10, wherein the p27 region is replaced with a linker region comprising the sequence set forth in SEQ ID NO. 2.

13. The HIV VLP of claim 10, wherein the fragment of RSV F protein comprises at least one point mutation selected from the group consisting of: S155C, S290C, S190F, and V207L.

14. A method of inducing an immune response against respiratory syncytial virus (RSV) in a subject, the method comprising administering the HIV VLP of claim 10 to the subject.

15. The method of claim 14, wherein the HIV VLP is administered at least twice and each administration is separated by at least three weeks.

16. The method of claim 14, wherein the HIV VLP is administered intranasally.

17. The method of claim 14, wherein mucosal immunity against RSV is induced.

18. The method of claim 14, wherein the HIV VLP is administered in a composition comprising the HIV VLP and an adjuvant.

19. A replicating geminiviral expression system for producing a human immunodeficiency virus (HIV) virus-like particle (VLP) comprising:
a first cloning vector with a T-DNA region comprising:
a sequence encoding Rep; and
a sequence encoding the promoter of ubiquitin-3 from potato with ubiquitin fusion;
a second cloning vector with a T-DNA region comprising:
a sequence encoding RepA; and
a sequence encoding the promoter of ubiquitin-3 from potato with ubiquitin fusion; and
a third cloning vector with a T-DNA region comprising an expression cassette and no replicon cassette, wherein the expression cassette comprises:
a promoter region;
a 5′ UTR;
a sequence encoding Gag;
a sequence encoding a fragment of gp41 and an antigen that is not a peptide or protein of HIV; and
a 3′ UTR.

20. The replicating geminiviral expression system of claim 19, wherein the expression level of the first cloning vector and the second cloning vector is 1:1.


SEQUENCE LISTING (26 sequences)

SEQ ID NO: 1 - protein, 6 aa, synthetic construct
  REGION 1..6: note = linker
GSGSGR

SEQ ID NO: 2 - protein, 181 aa, Human immunodeficiency virus 1
KIFIMIVGGLIGLRIIFAVLSMVNRVRQGYSPLSFQTLTPNPRGPDRLGRIEEEGGEQDRDRSIRLVSGFLALAWDDLRSLCLFSYHRLRDCILIVARAAELLGRSSLRGLQKGWEALKYLGSLVQYWGLELKKSAISLLDTTAIAVAEGTDRIIKLIQRICRAICNIPRRIRQGFEAALQ

SEQ ID NO: 3 - protein, 9 aa, synthetic construct
  REGION 1..9: note = linker
GSGSGRSLG

SEQ ID NO: 4 - DNA, 35 bp, synthetic construct
  misc_feature 1..35: note = primer
  source 1..35: mol_type = other DNA
aggggtctcgtggtcaaaacataaccgaggagttc

SEQ ID NO: 5 - DNA, 30 bp, synthetic construct
  misc_feature 1..30: note = primer
  source 1..30: mol_type = other DNA
ggggtctcataatgttagtggtggacttgc

SEQ ID NO: 6 - DNA, 35 bp, synthetic construct
  misc_feature 1..35: note = primer
  source 1..35: mol_type = other DNA
ggggtctccattaagattttcattatgattgtggg

SEQ ID NO: 7 - DNA, 26 bp, synthetic construct
  misc_feature 1..26: note = primer
  source 1..26: mol_type = other DNA
cttcttcttcttcttttctcattgtc

SEQ ID NO: 8 - DNA, 34 bp, synthetic construct
  misc_feature 1..34: note = primer
  source 1..34: mol_type = other DNA
ggggtctcgttacaatacaaaagtaaacccactg

SEQ ID NO: 9 - DNA, 32 bp, synthetic construct
  misc_feature 1..32: note = primer
  source 1..32: mol_type = other DNA
ggggtctcggtaacagagttaggcaaggatac

SEQ ID NO: 10 - DNA, 30 bp, synthetic construct
  misc_feature 1..30: note = primer
  source 1..30: mol_type = other DNA
gtggtctctgcaaacagcgacaccagaggc

SEQ ID NO: 11 - DNA, 33 bp, synthetic construct
  misc_feature 1..33: note = primer
  source 1..33: mol_type = other DNA
gtggtctcattgcaaagttctccatttggaagg

SEQ ID NO: 12 - DNA, 18 bp, synthetic construct
  misc_feature 1..18: note = primer
  source 1..18: mol_type = other DNA
cggcaacaggattcaatc

SEQ ID NO: 13 - DNA, 35 bp, synthetic construct
  misc_feature 1..35: note = primer
  source 1..35: mol_type = other DNA
gtggtctctacacattatgctatagctttgctgac

SEQ ID NO: 14 - DNA, 36 bp, synthetic construct
  misc_feature 1..36: note = primer
  source 1..36: mol_type = other DNA
gtggtctctgtgtatcatcaaagaagaagtgcttgc

SEQ ID NO: 15 - DNA, 34 bp, synthetic construct
  misc_feature 1..34: note = primer
  source 1..34: mol_type = other DNA
gtggtctctgaaggtcaaaactgaaactccatta

SEQ ID NO: 16 - DNA, 34 bp, synthetic construct
  misc_feature 1..34: note = primer
  source 1..34: mol_type = other DNA
gtggtctcccttcaaggtcttggaccttaagaac

SEQ ID NO: 17 - DNA, 35 bp, synthetic construct
  misc_feature 1..35: note = primer
  source 1..35: mol_type = other DNA
gtggtctcttgagtataggcaataactgtttgtca

SEQ ID NO: 18 - DNA, 39 bp, synthetic construct
  misc_feature 1..39: note = primer
  source 1..39: mol_type = other DNA
gtggtctcactcaacaagcaatcttgtagtattagcaac

SEQ ID NO: 19 - protein, 573 aa, synthetic construct
  REGION 1..573: note = plant-optimized RSV F protein
MANKHLSLSLFLVLLGLSASLASGQNITEEFYQSTCSAVSKGYLSALRTGWYTSVITIELSNIKENKCNGTDAKVKLIKQELDKYKNAVTELQLLTQSTPATNNRARRELPRFMNYTLNNAKKTNVTLSKKRKRRFLVFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 20 - protein, 551 aa, synthetic construct
  REGION 1..551: note = plant-optimized RSV F protein lacking p27 region
MANKHLSLSLFLVLLGLSASLASGQNITEEFYQSTCSAVSKGYLSALRTGWYTSVITIELSNIKENKCNGTDAKVKLIKQELDKYKNAVTELQLLTQSTPATNNRARGSGSGRSLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 21 - protein, 683 aa, synthetic construct
  REGION 1..683: note = HIV-like particle presenting RSV F protein
MANKHLSLSLFLVLLGLSASLASGQNITEEFYQSTCSAVSKGYLSALRTGWYTSVITIELSNIKENKCNGTDAKVKLIKQELDKYKNAVTELQLLTQSTPATNNRARGSGSGRSLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIKIFIMIVGGLIGLRIIFAVLSMVNRVRQGYSPLSFQTLTPNPRGPDRLGRIEEEGGEQDRDRSIRLVSGFLALAWDDLRSLCLFSYHRLRDCILIVARAAELLGRSSLRGLQKGWEALKYLGSLVQYWGLELKKSAISLLDTTAIAVAEGTDRIIKLIQRICRAICNIPRRIRQGFEAALQ

SEQ ID NO: 22 - protein, 551 aa, synthetic construct
  REGION 1..551: note = plant-optimized RSV F protein with pre-F stabilizing mutations
MANKHLSLSLFLVLLGLSASLASGQNITEEFYQSTCSAVSKGYLSALRTGWYTSVITIELSNIKENKCNGTDAKVKLIKQELDKYKNAVTELQLLTQSTPATNNRARGSGSGRSLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 23 - protein, 683 aa, synthetic construct
  REGION 1..683: note = HIV-like particle presenting RSV F protein with pre-F stabilizingmutations
MANKHLSLSLFLVLLGLSASLASGQNITEEFYQSTCSAVSKGYLSALRTGWYTSVITIELSNIKENKCNGTDAKVKLIKQELDKYKNAVTELQLLTQSTPATNNRARGSGSGRSLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIKIFIMIVGGLIGLRIIFAVLSMVNRVRQGYSPLSFQTLTPNPRGPDRLGRIEEEGGEQDRDRSIRLVSGFLALAWDDLRSLCLFSYHRLRDCILIVARAAELLGRSSLRGLQKGWEALKYLGSLVQYWGLELKKSAISLLDTTAIAVAEGTDRIIKLIQRICRAICNIPRRIRQGFEAALQ

SEQ ID NO: 24 - DNA, 15513 bp, synthetic construct
  misc_feature 1..15513: note = Expression vector pBYKEAM-BAF-gp41
  source 1..15513: mol_type = other DNA
cgatcggtcgattcatagaagattagatttttcatagtatttttttaaagtaaacctttaactacggttaggacacttttaagttaaatttaatttgaacccttaaattaatttttaaaatagataaatatcaatcatcctgatatgcttttgaaaaaatgaatgagaaagatgattcaattaaggccacattttaatcatgactaaaataatatacagtataatttcatatatatttgctttaaaaaaaaattgacaatccattcgtttctagcaataaatttcttcaaccacaaatatattaaagataactacggcatagaaacaaaaatctatgaagaatttttgtatacttcatatgaaattaaaaaaaacttcattgaacatcaaaataataataataatcataaactcctcaatatttatattcctagcttcttgaattaaattgtttacatattcaacgatgtaaaaaattatttctctatctattttccttatatcatgcatggtttcacatatatcaaaggataaaagcaatctatgtaaattatctcactttattaagttttctatctgaattattgagaacgtagatttctttttgcactatcccccaataattagcaaaacacacctagactagatttgttttgctaacccaattgatattaattatatatgattaatatttatatgtatatggaattggttaataaaatgcatctggttcatcaaagaattataaagacacgtgacattcatttaggataagaaatatggatgatctctttctcttattcagataattagtaattacacataacacacaactttgatgcccacattatagtgattagcatgtcactatgtgtgcatccttttatttcatacattaattaacttggccaatccagaagatggacaagtctagggtcacattgcagggtactctagcttactcgccttctttttcgaaggtttgagtaccttcagggcatcctcttgatacattactttccacttcgattggggcaagctgtagcagttcttgcttagaccgaattgccatctcacagagatgctgaagagttcgcgaccctccagaaacggtgatactaactcctcgaaaccgaatactataggtacatccgatctggtcgaaaccgaaaaatcgagatgctgcatagttaaccgaatctcccgtccaagatccaaggactctgtgcagtgaagcttccgtcctgtcgtatctgagatatctcttaaatacaactttcccgaaaccccagctttccttgaaaccaaggggattatcttgattcgaattcgtctcatcgttatgtagccgccactcagtccaactcggactttcgtcaggaagtttgaagggagaagttgtacctcctgatcctccatcccaacgttcactgttagcttgttccctagcgtcgtttccttgtatagctcgttccatggattgtaaatagtaattgtaatgttgtttgttgtttgttgttgttggtaattgttgtaaaaatacgctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctggcgcgccgctctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacgaccctcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagaaacaaacaaaatcaacaaatatagaaaataacgcatttccaattctttgaaatttctgcaacatctagaacaatggctaacaagcacctctcattgtctctcttccttgtgctccttggtctttctgcttctcttgcttctggtcaaaacataaccgaggagttctatcaaagtacttgttccgctgtttcaaaaggttacttaagtgcattacgtacaggctggtacactagtgttatcaccatagaactatcaaacattaaggagaacaaatgcaatggtacagatgcaaaggtcaaattgatcaaacaagagttggataagtataagaatgcagttacagagttacagttgttaacacaatctacacctgctacaaacaatcgagctaggagggagttgccaaggtttatgaattacacattgaataacgctaagaagacaaatgtgactctttccaagaagaggaagagaagatttctagtgttccttttgggagtcggtagtgccattgcctctggtgtcgctgtaagcaaagttctccatttggaaggagaagtcaacaagattaagtctgctcttctctcaactaacaaagctgttgttagtctgagtaatggagtttcagttttgacctcaaaggtcttggaccttaagaactacattgacaaacagttattgcctatagtcaataagcaatcttgtagtattagcaacatagaaacagttattgaatttcaacagaagaacaatcgtctactcgagataacaagagaattctccgtcaatgccggagtgactacaccagtatctacttacatgctaactaattctgaattgttaagtctcatcaatgatatgccaatcaccaatgaccaaaagaagctcatgtcaaacaatgttcaaattgttcgtcagcaaagctatagcataatgtctatcatcaaagaagaagtgcttgcatatgttgtacagcttcctctgtacggtgttatcgatactccatgttggaaactacatacttctcctctttgtacaacaaacacaaaagaggggagcaacatttgccttactagaactgatcgaggatggtattgtgataacgccgggtccgtttctttctttccccaggctgaaacatgcaaagtgcagtctaatagggtgttttgcgataccatgaatagtcttactttgccatccgaagtaaacctttgtaatgtggacattttcaatccaaagtatgattgcaaaatcatgacatctaagactgatgtgtcaagctcagtgattaccagtctgggagcaattgtttcatgctacggtaaaactaagtgtactgcttctaacaagaacagagggattatcaaaacttttagtaatggctgtgattatgtctctaacaaaggagtggataccgtttcagtaggcaatactttgtactacgtaaacaaacaagaaggaaagtcattgtatgttaaaggagagcctatcatcaatttctatgatcccctcgtatttccttctgacgagtttgatgcctctatatcacaagttaatgagaagatcaatcagagtctcgcattcataagaaaatccgatgagttattgcacaatgtgaatgctggcaagtccaccactaacattaagattttcattatgattgtgggaggattgattggattgaggatcattttcgctgtgttgtctatggtgaacagagttaggcaaggatactctcctttgtctttccaaactcttactccaaacccaaggggccctgataggttgggaaggattgaggaggagggaggagagcaagatagagataggtctattaggttggtgtctggattcttggctcttgcttgggatgatttgaggtctttgtgtcttttctcttaccataggttgagggattgtatcttgattgtggctagggctgctgagcttttgggaaggtcttctttgaggggccttcaaaagggatgggaggctcttaagtacttgggatctcttgtgcaatactggggacttgagcttaagaagtctgctatctctttgcttgatactaccgctattgctgtggctgagggcaccgataggatcatcaagttgattcaaaggatttgtagggcaatttgcaacatccctaggagaattagacaaggattcgaggctgctttgcaataagagctcaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgacgctcatacagcattcccagaaagagaaacagaagaaatatacaaactttcattttgagagcagcacctcgtctattgattgcagataatatgcttctcatttgtatttccttttgattatttttgtttctatccctttgtttgagtcaatctcaaatattcggtcattgttggtatgaaaaatcaagcagttcatgttaagagtcaatttaaaattaatatttttatatagagttgtatgtgaaatgatgttgtgatttggtatatatggataaagagcttgtcagttcattttggtctgatttttttggtatccaaataagaaacacaaaagggatatgtccctctactatcaaatattagttataagtattcatgttatactattcgatattttctaccccaatcgttacctatttaaaagtatttacccctccatctatcaaacccctggacccagctttcctattacatgtggcttcatcttaagcccccaaacctttttcttatttttgatttttaaaggctcatcttaaaatttattactcaaattaatacctcttaataacccacctcaaggacccagtaattaaatatccaattagctccagtaattggggttcatattagctccagtcttaaattttaaaggcgatgatcgtattcctccacttggttcatttatactcaaagaatactcaatgtctttagtgtttagataactttttgtaaatcatatagattgttttaacaaaaaacaattcaatagtagattttcacatgaaagttacataaaaattctttaaaattactttctcaaaaaattgttccaaacatattatcccacaattaaactcaatctgtttttcgaaacctaaatcaaaaccaatccaactaccttatataatatataatcaatacattgtaaagaactgcatgttcttttaaattttgggggcaaagttattccgtacgttcacacatgtactaataggaggtaataaatgatatgtgaaacaatcgaggtgtaaacaagctagcatgaattctcgattaaaaatcccaattatatttggtctaatttagtttggtattgagtaaaacaaattcgaaccaaaccaaaatataaatatatagtttttatatatatgcctttaagactttttatagaattttctttaaaaaatatctaggtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtggtaccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattgcaaagaattatagaagggattccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaagtcgatggtcccgttccagtagttgtgtcgcccgagacttctagcccaggtggtctttccggtacgagttggtccgcagatgtagaggctggggtgtctgaccccagtccttccctcatcctggttagatcggccatccactcaaggtcagattgtgcttgatcgtaggagacaggatgtatgaaagtgtaggcatcgatgcttacatgatataggtgcgtctctctccagttgtgcagatcttcgtggcagcggagatctgattctgtgaagggcgacacgtactgctcaggttgtggaggaaataatttgttggctgaatattccagccattgaagctttgttgcccattcatgagggaactcttctttgatcatgtcaagatactcctccttagacgttgcagtctggataatagttcgccatcgtgcgtcagatttgcgaggagacaccttatgatctcggaaatctcctctggttttaatatctccgtcctttgatatgtaatcaaggacttgtttagagtttctagctggctggatattagggtgatttccttcaaaatcgaaaaaagaaggatctctaatacaaggttttttatcaagctggataagagcatgatagtgggtagtgccatcttgatgaagctcagaagcaacaccaaggaagaaaataagaaaaggtgtgagtttctcccagagaaactggaataaatcatctctttgagatgagcacttggggtaggtaaggaaaacatatttagattggagtctgaagttcttgctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacggccggaccggtcccctaggccggccaattcgagatcggccgcggctgagtggctccttcaatcgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggagactgtcatacgcgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcgcaaaggcgctcggtcttgccttgctcgtcgagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaaggg

SEQ ID NO: 25 - DNA, 15447 bp, synthetic construct
  misc_feature 1..15447: note = Expression vector pBYKEAM-BAFd27-gp41
  source 1..15447: mol_type = other DNA
cgatcggtcgattcatagaagattagatttttcatagtatttttttaaagtaaacctttaactacggttaggacacttttaagttaaatttaatttgaacccttaaattaatttttaaaatagataaatatcaatcatcctgatatgcttttgaaaaaatgaatgagaaagatgattcaattaaggccacattttaatcatgactaaaataatatacagtataatttcatatatatttgctttaaaaaaaaattgacaatccattcgtttctagcaataaatttcttcaaccacaaatatattaaagataactacggcatagaaacaaaaatctatgaagaatttttgtatacttcatatgaaattaaaaaaaacttcattgaacatcaaaataataataataatcataaactcctcaatatttatattcctagcttcttgaattaaattgtttacatattcaacgatgtaaaaaattatttctctatctattttccttatatcatgcatggtttcacatatatcaaaggataaaagcaatctatgtaaattatctcactttattaagttttctatctgaattattgagaacgtagatttctttttgcactatcccccaataattagcaaaacacacctagactagatttgttttgctaacccaattgatattaattatatatgattaatatttatatgtatatggaattggttaataaaatgcatctggttcatcaaagaattataaagacacgtgacattcatttaggataagaaatatggatgatctctttctcttattcagataattagtaattacacataacacacaactttgatgcccacattatagtgattagcatgtcactatgtgtgcatccttttatttcatacattaattaacttggccaatccagaagatggacaagtctagggtcacattgcagggtactctagcttactcgccttctttttcgaaggtttgagtaccttcagggcatcctcttgatacattactttccacttcgattggggcaagctgtagcagttcttgcttagaccgaattgccatctcacagagatgctgaagagttcgcgaccctccagaaacggtgatactaactcctcgaaaccgaatactataggtacatccgatctggtcgaaaccgaaaaatcgagatgctgcatagttaaccgaatctcccgtccaagatccaaggactctgtgcagtgaagcttccgtcctgtcgtatctgagatatctcttaaatacaactttcccgaaaccccagctttccttgaaaccaaggggattatcttgattcgaattcgtctcatcgttatgtagccgccactcagtccaactcggactttcgtcaggaagtttgaagggagaagttgtacctcctgatcctccatcccaacgttcactgttagcttgttccctagcgtcgtttccttgtatagctcgttccatggattgtaaatagtaattgtaatgttgtttgttgtttgttgttgttggtaattgttgtaaaaatacgctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctggcgcgccgctctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacgaccctcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagaaacaaacaaaatcaacaaatatagaaaataacgcatttccaattctttgaaatttctgcaacatctagaacaatggctaacaagcacctctcattgtctctcttccttgtgctccttggtctttctgcttctcttgcttctggtcaaaacataaccgaggagttctatcaaagtacttgttccgctgtttcaaaaggttacttaagtgcattacgtacaggctggtacactagtgttatcaccatagaactatcaaacattaaggagaacaaatgcaatggtacagatgcaaaggtcaaattgatcaaacaagagttggataagtataagaatgcagttacagagttacagttgttaacacaatctacacctgctacaaacaatcgagctaggggatctggatctggtagatcactagggttccttttgggagtcggtagtgccattgcctctggtgtcgctgtaagcaaagttctccatttggaaggagaagtcaacaagattaagtctgctcttctctcaactaacaaagctgttgttagtctgagtaatggagtttcagttttgacctcaaaggtcttggaccttaagaactacattgacaaacagttattgcctatagtcaataagcaatcttgtagtattagcaacatagaaacagttattgaatttcaacagaagaacaatcgtctactcgagataacaagagaattctccgtcaatgccggagtgactacaccagtatctacttacatgctaactaattctgaattgttaagtctcatcaatgatatgccaatcaccaatgaccaaaagaagctcatgtcaaacaatgttcaaattgttcgtcagcaaagctatagcataatgtctatcatcaaagaagaagtgcttgcatatgttgtacagcttcctctgtacggtgttatcgatactccatgttggaaactacatacttctcctctttgtacaacaaacacaaaagaggggagcaacatttgccttactagaactgatcgaggatggtattgtgataacgccgggtccgtttctttctttccccaggctgaaacatgcaaagtgcagtctaatagggtgttttgcgataccatgaatagtcttactttgccatccgaagtaaacctttgtaatgtggacattttcaatccaaagtatgattgcaaaatcatgacatctaagactgatgtgtcaagctcagtgattaccagtctgggagcaattgtttcatgctacggtaaaactaagtgtactgcttctaacaagaacagagggattatcaaaacttttagtaatggctgtgattatgtctctaacaaaggagtggataccgtttcagtaggcaatactttgtactacgtaaacaaacaagaaggaaagtcattgtatgttaaaggagagcctatcatcaatttctatgatcccctcgtatttccttctgacgagtttgatgcctctatatcacaagttaatgagaagatcaatcagagtctcgcattcataagaaaatccgatgagttattgcacaatgtgaatgctggcaagtccaccactaacattaagattttcattatgattgtgggaggattgattggattgaggatcattttcgctgtgttgtctatggtgaacagagttaggcaaggatactctcctttgtctttccaaactcttactccaaacccaaggggccctgataggttgggaaggattgaggaggagggaggagagcaagatagagataggtctattaggttggtgtctggattcttggctcttgcttgggatgatttgaggtctttgtgtcttttctcttaccataggttgagggattgtatcttgattgtggctagggctgctgagcttttgggaaggtcttctttgaggggccttcaaaagggatgggaggctcttaagtacttgggatctcttgtgcaatactggggacttgagcttaagaagtctgctatctctttgcttgatactaccgctattgctgtggctgagggcaccgataggatcatcaagttgattcaaaggatttgtagggcaatttgcaacatccctaggagaattagacaaggattcgaggctgctttgcaataagagctcaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgacgctcatacagcattcccagaaagagaaacagaagaaatatacaaactttcattttgagagcagcacctcgtctattgattgcagataatatgcttctcatttgtatttccttttgattatttttgtttctatccctttgtttgagtcaatctcaaatattcggtcattgttggtatgaaaaatcaagcagttcatgttaagagtcaatttaaaattaatatttttatatagagttgtatgtgaaatgatgttgtgatttggtatatatggataaagagcttgtcagttcattttggtctgatttttttggtatccaaataagaaacacaaaagggatatgtccctctactatcaaatattagttataagtattcatgttatactattcgatattttctaccccaatcgttacctatttaaaagtatttacccctccatctatcaaacccctggacccagctttcctattacatgtggcttcatcttaagcccccaaacctttttcttatttttgatttttaaaggctcatcttaaaatttattactcaaattaatacctcttaataacccacctcaaggacccagtaattaaatatccaattagctccagtaattggggttcatattagctccagtcttaaattttaaaggcgatgatcgtattcctccacttggttcatttatactcaaagaatactcaatgtctttagtgtttagataactttttgtaaatcatatagattgttttaacaaaaaacaattcaatagtagattttcacatgaaagttacataaaaattctttaaaattactttctcaaaaaattgttccaaacatattatcccacaattaaactcaatctgtttttcgaaacctaaatcaaaaccaatccaactaccttatataatatataatcaatacattgtaaagaactgcatgttcttttaaattttgggggcaaagttattccgtacgttcacacatgtactaataggaggtaataaatgatatgtgaaacaatcgaggtgtaaacaagctagcatgaattctcgattaaaaatcccaattatatttggtctaatttagtttggtattgagtaaaacaaattcgaaccaaaccaaaatataaatatatagtttttatatatatgcctttaagactttttatagaattttctttaaaaaatatctaggtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtggtaccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattgcaaagaattatagaagggattccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaagtcgatggtcccgttccagtagttgtgtcgcccgagacttctagcccaggtggtctttccggtacgagttggtccgcagatgtagaggctggggtgtctgaccccagtccttccctcatcctggttagatcggccatccactcaaggtcagattgtgcttgatcgtaggagacaggatgtatgaaagtgtaggcatcgatgcttacatgatataggtgcgtctctctccagttgtgcagatcttcgtggcagcggagatctgattctgtgaagggcgacacgtactgctcaggttgtggaggaaataatttgttggctgaatattccagccattgaagctttgttgcccattcatgagggaactcttctttgatcatgtcaagatactcctccttagacgttgcagtctggataatagttcgccatcgtgcgtcagatttgcgaggagacaccttatgatctcggaaatctcctctggttttaatatctccgtcctttgatatgtaatcaaggacttgtttagagtttctagctggctggatattagggtgatttccttcaaaatcgaaaaaagaaggatctctaatacaaggttttttatcaagctggataagagcatgatagtgggtagtgccatcttgatgaagctcagaagcaacaccaaggaagaaaataagaaaaggtgtgagtttctcccagagaaactggaataaatcatctctttgagatgagcacttggggtaggtaaggaaaacatatttagattggagtctgaagttcttgctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacggccggaccggtcccctaggccggccaattcgagatcggccgcggctgagtggctccttcaatcgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggagactgtcatacgcgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcgcaaaggcgctcggtcttgccttgctcgtcgagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaaggg

SEQ ID NO: 26 - DNA, 15167 bp, synthetic construct
  misc_feature 1..15167: note = Expression vector pBYKEAM-gag
  source 1..15167: mol_type = other DNA
cgatcggtcgattcatagaagattagatttttcatagtatttttttaaagtaaacctttaactacggttaggacacttttaagttaaatttaatttgaacccttaaattaatttttaaaatagataaatatcaatcatcctgatatgcttttgaaaaaatgaatgagaaagatgattcaattaaggccacattttaatcatgactaaaataatatacagtataatttcatatatatttgctttaaaaaaaaattgacaatccattcgtttctagcaataaatttcttcaaccacaaatatattaaagataactacggcatagaaacaaaaatctatgaagaatttttgtatacttcatatgaaattaaaaaaaacttcattgaacatcaaaataataataataatcataaactcctcaatatttatattcctagcttcttgaattaaattgtttacatattcaacgatgtaaaaaattatttctctatctattttccttatatcatgcatggtttcacatatatcaaaggataaaagcaatctatgtaaattatctcactttattaagttttctatctgaattattgagaacgtagatttctttttgcactatcccccaataattagcaaaacacacctagactagatttgttttgctaacccaattgatattaattatatatgattaatatttatatgtatatggaattggttaataaaatgcatctggttcatcaaagaattataaagacacgtgacattcatttaggataagaaatatggatgatctctttctcttattcagataattagtaattacacataacacacaactttgatgcccacattatagtgattagcatgtcactatgtgtgcatccttttatttcatacattaattaacttggccaatccagaagatggacaagtctagggtcacattgcagggtactctagcttactcgccttctttttcgaaggtttgagtaccttcagggcatcctcttgatacattactttccacttcgattggggcaagctgtagcagttcttgcttagaccgaattgccatctcacagagatgctgaagagttcgcgaccctccagaaacggtgatactaactcctcgaaaccgaatactataggtacatccgatctggtcgaaaccgaaaaatcgagatgctgcatagttaaccgaatctcccgtccaagatccaaggactctgtgcagtgaagcttccgtcctgtcgtatctgagatatctcttaaatacaactttcccgaaaccccagctttccttgaaaccaaggggattatcttgattcgaattcgtctcatcgttatgtagccgccactcagtccaactcggactttcgtcaggaagtttgaagggagaagttgtacctcctgatcctccatcccaacgttcactgttagcttgttccctagcgtcgtttccttgtatagctcgttccatggattgtaaatagtaattgtaatgttgtttgttgtttgttgttgttggtaattgttgtaaaaatacgctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctggcgcgccgctctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacgaccctcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagaaacaaacaaaatcaacaaatatagaaaataacgcatttccaattctttgaaatttctgcaacatctagaacaatgggagctagagcctctatcttgaggggagagaagttggacaagtgggagaagattaggttgaggccaggaggcaagaagcactacatgctcaagcacttggtgtgggctagtagggagcttgagaggtttgctctcaaccctggcctcttagagacaagtgagggctgtaagcaaatcatgaagcaacttcaaccagctctccaaacaggaacagaggagcttaggtctctctacaacacagtggctactctttgttgtgtgcatgagaagattgaggtgagggacaccaaggaggccttggacaagatggaggaggagcaaaacaagagccaacaaaagacacaacaagctgaggctgctgacaagggaaaggttagccaaaactaccctattgttcaaaacctccaaggacaaatggtgcaccaagccatctctccaaggactctcaatgcttgggtgaaggtgattgaggagaaggcttttagcccagaggtgatccctatgttcacagctttgagtgagggagccaccccacaagacttgaacaccatgttgaacacagtgggaggacaccaagctgccatgcaaatgttgaaggacaccatcaatgaggaggctgctgagtgggataggttgcacccagtgcatgctggacctattgctccaggccaaattagggagccaaggggaagtgacattgctggaactactagcaccctccaagagcaaattgcttggatgacaaacaacccacctatcccagtgggagatatctacaagagatggattattcttggattgaacaagattgtgaggatgtatagccctgttagcatcttggacattaagcaaggaccaaaggagccttttagggactatgtggataggttcttcaagactttgagggctgagcaatctacacaagaggtgaagaattggatgacagacaccttgttggttcaaaatgccaacccagattgtaagaccatcttgagggctttgggcacaggagctacattggaggagatgatgacagcttgtcaaggagtgggaggacctagccacaaggctagggttttggctgaggctatgagccaagctggcaatacaaacattatgatgcaaaggagcaacttcaagggccctagaaggatcattaagtgtttcaattgtggcaaggagggacacattgctaggaattgtagggcccctaggaagaaggggtgttggaagtgtggaaaggagggacaccaaatcaaggattgtactgagaggcaagctaacttcttgggaaagatttggccttcccacaagggaaggccaggaaatttcctccaaaacaggccagagccaacagccccaccagctgagagctttaggtttgaagagacaacccctgctccaaagcaagagccaaatgatagggagcctttgacttccctcaagtctctctttggaagtgacccttcctctcaataagagctcaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgacggctcatacagcattcccagaaagagaaacagaagaaatatacaaactttcattttgagagcagcacctcgtctattgattgcagataatatgcttctcatttgtatttccttttgattatttttgtttctatccctttgtttgagtcaatctcaaatattcggtcattgttggtatgaaaaatcaagcagttcatgttaagagtcaatttaaaattaatatttttatatagagttgtatgtgaaatgatgttgtgatttggtatatatggataaagagcttgtcagttcattttggtctcatttttttggtatccaaataagaaacacaaaagggatatgtccctctactatcaaatattagttataagtattcatgttatactattcgatattttctaccccaatcgttacctatttaaaagtatttacccctccatctatcaaacccctggacccagctttcctattacatgtggcttcatcttaagcccccaaacctttttcttatttttgatttttaaaggctcatcttaaaatttattactcaaattaatacctcttaataacccacctcaaggacccagtaattaaatatccaattagctccagtaattggggttcatattagctccagtcttaaattttaaaggcgatgatcgtattcctccacttggttcatttatactcaaagaatactcaatgtctttagtgtttagataactttttgtaaatcatatagattgttttaacaaaaaacaattcaatagtagattttcacatgaaagttacataaaaattctttaaaattactttctcaaaaaattgttccaaacatattatcccacaattaaactcaatctgtttttcgaaacctaaatcaaaaccaatccaactaccttatataatatataatcaatacattgtaaagaactgcatgttcttttaaattttgggggcaaagttattccgtacgttcacacatgtactaataggaggtaataaatgatatgtgaaacaatcgaggtgtaaacaagctagcatgaattctcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtggtaccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaagtcgatggtcccgttccagtagttgtgtcgcccgagacttctagcccaggtggtctttccggtacgagttggtccgcagatgtagaggctggggtgtctgaccccagtccttccctcatcctggttagatcggccatccactcaaggtcagattgtgcttgatcgtaggagacaggatgtatgaaagtgtaggcatcgatgcttacatgatataggtgcgtctctctccagttgtgcagatcttcgtggcagcggagatctgattctgtgaagggcgacacgtactgctcaggttgtggaggaaataatttgttggctgaatattccagccattgaagctttgttgcccattcatgagggaactcttctttgatcatgtcaagatactcctccttagacgttgcagtctggataatagttcgccatcgtgcgtcagatttgcgaggagacaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggataagagcatgatagtgggtagtgccatcttgatgaagctcagaagcaacaccaaggaagaaaataagaaaaggtgtgagtttctcccagagaaactggaataaatcatctctttgagatgagcacttggggtaggtaaggaaaacatatttagattggagtctgaagttcttgctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacggccggaccggtcccctaggccggccaattcgagatcggccgcggctgagtggctccttcaatcgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggagactgtcatacgcgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcgcaaaggcgctcggtcttgccttgctcgtcgagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaaggg